(19) United States
(12) Patent Application Publication
(10) Pub. No.: US 2026/0158236 A1
(43) Pub. Date: Jun. 11, 2026

(54) SMELL PRESENTATION AND THERAPEUTIC USE

(71) Applicants: Monell Chemical Senses Center, Philadelphia, PA (US); Ahersla Health, Inc., Long Beach Township, NJ (US)

(72) Inventors: Valentina PARMA, Philadelphia, PA (US); Patricia LUCAS-SCHNARRE, Long Beach Township, NJ (US); Pamela H. DALTON, Philadelphia, PA (US)

(21) Appl. No.: 19/362,879

(22) Filed: Oct. 20, 2025

Related U.S. Application Data
(63) Continuation of application No. PCT/US2024/025505, filed on Apr. 19, 2024.
(60) Provisional application No. 63/497,659, filed on Apr. 21, 2023.

(51) Int. Cl. A61M 21/02; A61B 5/00; A61B 5/16; A61M 21/00

(52) U.S. Cl. CPC A61M 21/02; A61B 5/165; A61B 5/4011; A61B 5/4848; A61B 5/742; A61B 5/7475; A61M 2021/0016; A61M 2205/502; A61M 2205/583

(57) ABSTRACT

Provided are systems, methods, and kits for multifunctional smell presentation. The kits may comprise a panel comprising a plurality of patches comprising distinct odorous volatile compounds, images indicating the source of the odorous volatile compounds, and labels indicating the identity of the odorous volatile compounds. The kits may comprise a system for receiving a plurality of inputs from a user. The system or methods may comprise receiving inputs from a user on whether the user detects the odorous volatile compounds, identifies the source of the odorous volatile compounds, and a rating on the intensity of the odorous volatile compounds.

### CROSS-REFERENCE

This application is a continuation of International Application No. PCT/US2024/025505, filed on Apr. 19, 2024, which claims the benefit of U.S. Provisional Application No. 63/497,659, entitled “SMELL PRESENTATION AND THERAPEUTIC USE”, filed on Apr. 21, 2023, which is incorporated by reference herein in its entirety.

### STATEMENT AS TO FEDERALLY SPONSORED RESEARCH

This invention was made with government support under Grant ID U01DC019578 awarded by the National Institute on Deafness and Other Communication Disorders (NIDCD), a member of the U.S. National Institutes of Health. The government has certain rights in the invention.

### SUMMARY

In one aspect, provided herein is a method for improving or changing a user's mental state, comprising providing the user a panel comprising: a plurality of patches, each of the plurality of patches comprising odorous volatile compound having a prescribed distinct odor. In some aspects, the subject performs an odor search among the plurality of patches, or is instructed to perform the odor search. In some aspects, the test administrator will communicate instructions for an odor search. In another aspect, provided herein is a method for improving or changing a user's mental state, comprising providing the user a panel comprising: a plurality of patches, each of the plurality of patches comprising odorous volatile compound having a prescribed distinct odor, and periodically quantitatively and/or qualitatively assessing the user's ability to correctly characterize the prescribed distinct odor, thereby assessing the user's mental state.

In one aspect, provided herein is a method for improving or changing a user's mental state, comprising: a) providing the user a panel comprising: i) a plurality of patches, each of the plurality of patches comprising odorous volatile compound having a prescribed distinct odor, and ii) labels or images on, or adjacent to, the plurality of patches, wherein the images or labels are indicative of the prescribed distinct odor; and b) periodically quantitatively and/or qualitatively assessing the user's ability to correctly characterize the prescribed distinct odor, thereby assessing the user's mental state. In some embodiments, the subject performs an odor search among the plurality of patches, or is instructed to perform the odor search. In some embodiments, the method further comprises an input, wherein the input comprises a rating on an intensity of the prescribed distinct odor of each of the plurality of patches as perceived by the user. In some embodiments, the user is suffering from PTSD, anxiety, or a negative mental state. In some embodiments, the user is suffering from a loss of smell. In some embodiments, the user is suffering from a loss of smell, optionally wherein the loss of smell is a result of an infection. In some embodiments, the infection is Severe Acute Respiratory Syndrome Coronavirus 2 (SARS CoV-2). In some embodiments, the method further comprises, c) instructing the user to complete a smell presentation test by using a computer, optionally wherein the computer is a portable electronic device. In some embodiments, a portable electronic device can be a phone, for example a touch phone. In some embodiments, the computer instructs the user to complete the smell presentation test at least twice daily over the course of at least eight weeks. In some embodiments, the method further comprises, d) storing a plurality of inputs each time the plurality of inputs is received from the computer. In some embodiments, the method further comprises, e) determining, whether the user's mental state has improved based on the plurality of inputs. In some embodiments, the periodically quantitatively and/or qualitatively assessing the user's ability to correctly characterize the prescribed distinct odor further comprises receiving additional input from the user, the additional input comprising: (a) input on the user's physiological data, (b) input on the user's mood, (c) input of the user's mental health status, or (d) a combination of any one thereof. In some embodiments, the method comprises assessing the user's mental state after input on the user's physiological data. In some embodiments, the method comprises assessing the user's mental state after input on the user's mood. In some embodiments, the method comprises assessing the user's mental state after input of the user's mental health status. In some embodiments, the method comprises determining whether the user's mental state has improved compared to a placebo or compared to the user's mental state prior to input on the user's physiological data. In some embodiments, the method further comprises, assessing the user's mental state after periodically quantitatively and/or qualitatively assessing the user's ability to correctly characterize the prescribed distinct odor. In some embodiments, the method further comprises, assessing the user's mental state after instructing the user to complete a smell presentation test by using a computer. In some embodiments, the method further comprises, assessing the user's mental state after the user to completes the smell presentation test at least twice daily over the course of at least eight weeks. In some embodiments, the method further comprises, assessing the user's mental state after d). In some embodiments, the method further comprises, determining whether the user's mental state has improved compared to a placebo or compared to the user's mental state prior to providing the user a panel. In some embodiments, the method further comprises providing a therapeutic volatile odorous compound to the user. In some embodiments, the method further comprises administration or recommendation of a therapeutic agent to the user. In some embodiments, in view of a result, the method comprises recommendation of a treatment. In some embodiments, in view of a result the method comprises recommendation of a therapeutic agent. In some embodiments, the method further comprises assessing a user's time lapse in correctly characterizing the prescribed distinct odor. In some embodiments, the user performs an odor search among the plurality of patches to momentarily change a mental state in order to provide a cognitive and/or behavioral therapy.

In some aspects, provided herein is a smell presentation system, comprising: a) a plurality of panels, each panel comprising: i) a plurality of odorous volatile compounds, each separately adhered to one panel on the plurality of panels and to a cover on each panel comprising a distinct odor; and ii) an image or label located adjacent to each of the plurality of odorous volatile compounds or on each cover, wherein the image or label is indicative of the distinct odor of each odorous volatile compound; and b) an electronic system, comprising an output device that prompts a user to detect, by olfaction, the plurality of odorous volatile compounds at least two times per day, and comprising an input device that registers an input from the user when the user attempts to identify each odorous volatile compound. In some embodiments, the input device obtains data from the user on whether the user detects, by olfaction, each of the plurality of odorous volatile compounds. In some embodiments, the input device obtains data from the user on whether the user identifies two or more distinct odors of the plurality of odorous volatile compounds. In some embodiments, the input device obtains data comprises a rating from the user on an intensity of the distinct odor of each of the plurality of odorous volatile compounds. In some embodiments, the electronic system compares the data to a reference. In some embodiments, the reference is a control. In some embodiments, the output device presents the data to a peer group. In some embodiments, a peer group can comprise a medical profession. In some embodiments, a prompt by the output device is gamified. In some embodiments, the electronic system receives data at least twice daily over the course of at least eight weeks. In some embodiments, the electronic system further comprises a storage device that stores data each time data is received. In some embodiments, the system further comprises a processor that determines whether the user's mental state improves over time. In some embodiments, each cover comprises plastic, rubber, silicone, or paper. In some embodiments, each cover is transparent. In some embodiments, each panel comprises paper, card stock, plastic, rubber, metal, or silicone. In some embodiments, the input device provides a psychological questionnaire to a user.

In some aspects, provided herein is a method for improving a user's sense of smell, comprising: a) providing the user a plurality of panels, each panel of the plurality of panels comprising: i) a plurality of patches, each of the plurality of patches comprising a distinct odorous volatile compound adhered to the panel, and ii) an image or label located adjacent to or on each of the plurality of patches, wherein the image or label is indicative of the odor of each patch; and b) exposing the user to the odorous volatile compounds of the plurality of patches of a first panel of the plurality of panels at least twice daily over at least eight weeks. In some embodiments, the method further comprises exposing the user to the odorous volatile compounds of the plurality of patches of a second panel of the plurality of panels twice daily over eight weeks. In some embodiments, the method further comprises exposing the user to the odorous volatile compounds of the plurality of patches of a third panel of the plurality of panels twice daily over eight weeks. In some embodiments, the method further comprises an input on whether the user identifies the source or the odorous volatile compound. In some embodiments, the method further comprises an input comprising a rating on an intensity of the odor of each odorous volatile compound as perceived by the user. In some embodiments, the user is suffering from PTSD, anxiety, and/or a psychological disease. In some embodiments, the user is suffering from a loss of smell as a result of an infection. In some embodiments, the infection is Severe Acute Respiratory Syndrome Coronavirus 2 (SARS-CoV-2). In some embodiments, the method further comprises, c) instructing the user to complete a smell presentation test comprising the plurality of panels. In some embodiments, the smell presentation test is completed by the user twice daily over the course of eight weeks. In some embodiments, the method further comprises, d) storing the plurality of inputs each time the plurality of inputs is received. In some embodiments, the method further comprises, e) determining, based on the user's inputs, whether the user's mental state has improved. In some embodiments, b) further comprises receiving additional input from the user, the additional input comprising: (a) input on the user's physical health, (b) input on the user's mood, (c) input of the user's mental health status, or (d) a combination thereof. In some embodiments, the method comprises assessing the user's mental state after input on the user's physical health. In some embodiments, the method comprises assessing the user's mental state after input on the user's mood. In some embodiments, the method comprises assessing the user's mental state after input of the user's mental health status. In some embodiments, the method comprises determining whether the user's mental state has improved compared to a placebo or compared to the user's mental state prior to input on the user's physical health. In some embodiments, the method further comprises, assessing the user's mental state after exposing the user to the odorous volatile compounds of the plurality of patches. In some embodiments, the method further comprises, assessing the user's mental state after completing a smell presentation test comprising the plurality of panels. In some embodiments, the method further comprises, assessing the user's mental state after the user to completes the smell presentation test at least twice daily over the course of at least eight weeks. In some embodiments, the method further comprises, assessing the user's mental state after d). In some embodiments, the method further comprises, determining whether the user's mental state has improved compared to a placebo or compared to the user's mental state prior to providing the user a plurality of panels. In some embodiments, the user performs an odor search among the plurality of patches to momentarily change a mental state in order to provide a cognitive and/or behavioral therapy.

In another aspect, provided herein is a computer-implemented method for assessing a user's mental state, comprising: a) providing the user a panel comprising: i) a plurality of patches adhered to the panel, each of the plurality of patches comprising a distinct odorous volatile compound; and ii) an image or a label located adjacent to or on each of the plurality of patches, wherein the image or the label is indicative of the odor; and b) receiving, by a computer, a first plurality of inputs from the user via a smell presentation test, the first plurality of inputs comprising: (i) a first input on whether the user detects, by olfaction, the odorous volatile compounds; (ii) a second input on whether the user identifies the source of the odorous volatile compounds; and (iii) a third input comprising a rating on an intensity of the odor of each distinct odorous volatile compound as perceived by the user; wherein (i), (ii), and (iii) may be in any order; and c) instructing, by the computer, the user to complete the smell presentation test about twice daily over about eight weeks. In some embodiments, the user detects, by olfaction, the odorous volatile compound. In some embodiments, the user identifies the source of the odorous volatile compounds. In some embodiments, the user identifies with a rating on the intensity of the odor of each odorous volatile compound as perceived by the user. In some embodiments, the user is suffering from PTSD, anxiety, depression, and/or a psychological disorder. In some embodiments, the user is suffering from obesity, and/or an eating disorder. In some embodiments, the user is suffering from a loss of smell as a result of an infection. In some embodiments, the infection is Severe Acute Respiratory Syndrome Coronavirus 2 (SARS-CoV-2). In some embodiments, the method further comprises, d) instructing, by the computer, the user to complete the smell presentation test. In some embodiments, the computer instructs the user to complete the smell presentation test twice daily over the course of eight weeks. In some embodiments, the computer instructs the user to complete the smell presentation test having a short exposure or a prolonged exposure, such as a 20-min exposure or shorter exposures over 5 consecutive days. In some embodiments, the method further comprises, e) storing, by the computer, the plurality of inputs each time the plurality of inputs is received. In some embodiments, the method further comprises, f) determining, based on the user's inputs, whether the user's mental state has improved. In some embodiments, the method further comprises, f) determining, based on the user's inputs, whether the user has experienced a decrease in appetite and/or weight loss. In some embodiments, b) further comprises receiving, by the computer, additional input from the user, the additional input comprising: (a) input on the user's physical health, (b) input on the user's mood, (c) input of the user's mental health status, or (d) a combination thereof. In some embodiments, the method comprises assessing the user's mental state after input on the user's physical health. In some embodiments, the method comprises assessing the user's mental state after input on the user's mood. In some embodiments, the method comprises assessing the user's mental state after input of the user's mental health status. In some embodiments, the method comprises determining whether the user's mental state has improved compared to a placebo or compared to the user's mental state prior to input on the user's physical health. In some embodiments, the method further comprises, assessing the user's mental state after the user detects, by olfaction, the odorous volatile compounds and/or after the user identifies the source of the odorous volatile compounds. In some embodiments, the method further comprises, assessing the user's mental state after the user rates the intensity of the odor of each distinct odorous volatile compound as perceived by the user. In some embodiments, the method further comprises, assessing the user's mental state after the user to completes the smell presentation test at least twice daily over the course of at least eight weeks. In some embodiments, the method further comprises, assessing the user's mental state after e). In some embodiments, the method further comprises, determining whether the user's mental state has improved compared to a placebo or compared to the user's mental state prior to providing the user a panel.

In another aspect, provided herein is a multifunctional smell presentation tool comprising: i) one or more lift-and-smell odor panels comprising one or more odorous volatile compounds, wherein the one or more odorous volatile compounds ranges in concentration; and ii) a system for instructing a user to interact with the one or more lift-and-smell odor panels based on instructions to induce the user to identify a specific odor amongst one or more distraction odors.

Provided herein, in some aspects, are methods for maintaining neurological function using the multifunctional smell presentation tools provided herein, wherein a test administrator further communicates instructions to prolong or repeat odor exposure. Provided herein, in some aspects, are methods for enhancing neurological function comprising cognition or memory using the multifunctional smell presentation tools and/or the smell presentation systems provided herein, wherein a test administrator further communicates instructions to prolong or repeat odor exposure. Provided herein, in some aspects, are methods for influencing eating behavior and diet quality or nutritional intake using the multifunctional smell presentation tools and/or the smell presentation systems provided herein, wherein a test administrator further communicates instructions to prolong or repeat odor exposure.

Provided herein, in some aspects, is a multifunctional smell presentation tool comprising i) one or more lift-and-smell odor panels comprising one or more odorous volatile compounds; and ii) a system for instructing a user to interact with the one or more lift-and-smell odor panels. In some embodiments, the one or more lift-and-smell odor panels comprises a cover. In some embodiments, the cover comprises plastic, rubber, silicone, or paper. In some embodiments, the cover is transparent. In some embodiments, the one or more lift-and-smell odor panels comprises paper, card stock, plastic, rubber, metal, or silicone. In some embodiments, the one or more odorous volatile compounds comprises odors of healthy food items, such as fresh food and vegetables. In some embodiments, the one or more odorous volatile compounds comprises odors of indulging food items, such as chocolate. In some embodiments, the one or more odorous volatile compounds ranges in concentration. In some embodiments, the one or more odorous volatile compounds has a concentration of about 0.25 mg/mL to about 1 mg/mL. In some embodiments, the system for instructing a user to interact with the one or more lift-and-smell odor panels comprises instructions to induce the user to identify a specific odor amongst one or more distraction odors.

Provided herein, in some aspects, are methods for maintaining neurological function using the multifunctional smell presentation tools and/or the smell presentation systems provided herein, wherein a test administrator further communicates instructions to prolong or repeat odor exposure.

Provided herein, in some aspects, is a method for promoting weight loss in a user, comprising: a) providing the user a panel, wherein the panel comprises: i) a patch, wherein the patch comprises an odorous volatile compound adhered to the panel, and ii) an image or label located adjacent to, or on, the patch, wherein the image or label is indicative of the odor of the patch; and b) exposing the user to the odorous volatile compound of the patch. In some embodiments, the panel comprises a plurality of panels. In some embodiments, the patch comprises a plurality of patches. In some embodiments, the method further comprises exposing the user to the odorous volatile compound for a short exposure time, such as less than 5 minutes. In some embodiments, the method further comprises exposing the user to the odorous volatile compound for a prolonged exposure time, such as a 20-min exposure or shorter exposures over 5 consecutive days. In some embodiments, the method further comprises an input on whether the user identifies the source or the odorous volatile compound. In some embodiments, the method further comprises an input comprising a rating on an intensity of the odor of each odorous volatile compound as perceived by the user. In some embodiments, the method further comprises, c) instructing the user to complete a smell presentation test. In some embodiments, the method further comprises, d) storing the input each time the input is received. In some embodiments, the method further comprises, e) determining, based on the input, whether the user's body weight has decreased. In some embodiments, b) further comprises receiving additional input from the user, the additional input comprising: (a) input on the user's physical health, (b) input on the user's mood, (c) input of the user's mental health status, or (d) a combination thereof. In some embodiments, the method comprises assessing the user's body weight after input on the user's physical health. In some embodiments, the method comprises assessing the user's body weight after input on the user's physical health. In some embodiments, the method comprises assessing the user's body weight after input on the user's mood. In some embodiments, the method comprises assessing the user's body weight after input of the user's mental health status. In some embodiments, the method comprises determining whether the user's body weight has improved compared to a placebo or compared to the user's body weight prior to input on the user's physical health. In some embodiments, the method further comprises, assessing the user's body weight after exposing the user to the odorous volatile compound of the patch. In some embodiments, the method further comprises, assessing the user's body weight after the user completes a smell presentation test. In some embodiments, the method further comprises, assessing the user's body weight after exposing the user to the odorous volatile compound for a short exposure time, such as less than 5 minutes. In some embodiments, the method further comprises, assessing the user's body weight after exposing the user to the odorous volatile compound for a prolonged exposure time, such as a 20-min exposure or shorter exposures over 5 consecutive days. In some embodiments, the method further comprises, assessing the user's body weight after the user to completes the smell presentation test at least twice daily over the course of at least eight weeks. In some embodiments, the method further comprises, assessing the user's body weight after d). In some embodiments, the method further comprises, determining whether the user's body weight has improved compared to a placebo or compared to the user's body weight prior to providing the user a panel. In some embodiments, the user performs an odor search among the plurality of patches to momentarily change a mental state in order to provide a cognitive and/or behavioral therapy.

### BRIEF DESCRIPTION OF THE DRAWINGS

FIG. 1A-1C show examples of a smell presentation kit.

FIG. 2 shows various components that may be included in a smell presentation kit.

FIG. 3 shows a schematic illustration of a panel of a multifunctional smell assessment kit with a releasable adhesive.

FIG. 4 shows various components of a smell presentation kit.

FIG. 5 shows various components of a smell presentation kit.

FIG. 6 is a diagram of a computer system that may be programmed or otherwise configured to implement methods provided herein.

FIG. 7 depicts standard user experience (UX) criteria measured using the User Experience Questionnaire (UEQ). N=37 adults.

FIG. 8 shows stability of odor intensity, as reported daily on a 0-100 scale, over 14 days for a group of N=8 adults with normal olfactory function.

FIG. 9 shows various components of a smell presentation kit for odor delivery for olfactory nudge (ON) and olfactory habituation (OH) conditions.

FIG. 10 depicts weight change over two weeks of exposure to olfactory nudge (ON) strategy, olfactory habituation (OH) strategy, and no odor (control condition).

FIG. 11 shows olfactory awareness based on olfactory exposure to olfactory nudge (ON) strategy, olfactory habituation (OH) strategy, and no odor (control condition).

### DETAILED DESCRIPTION

People suffer from PTSD (post-traumatic stress disorder) after experiencing a range of events: war, victim of crime, natural disaster, abuse, an accident, being a witness to trauma and more. It is a stress-based condition resulting in significant changes in cognitive function (memory, attention, problem solving) and emotional regulation. The American Psychiatric Association (APA) reports that 3.5% of adults are suffering from PTSD each year. Obsessive-compulsive disorder (OCD) affects 2.2 million adults according to the Anxiety & Depression Association of America(ADAA). The defining characteristic of OCD is intrusive thoughts and fears that lead to repetitive behaviors. Interestingly, 94% of people experience some form of obsessive thoughts at some point in time according to a study by Radomsky published in the Journal of Obsessive-Compulsive and Related Disorders. Obsessive thoughts can also occur in post-partum depression and eating disorders. One way to manage obsessive thought is through redirecting to something that grabs the attention securely—like evaluating wine by sniff to identify its characteristics and quality or possibly by detecting aromas that are familiar but not clearly identifiable. According to APA the criteria for diagnosis of PTSD includes re-experiencing (described as spontaneous memories of the traumatic event, related recurrent dreams, flashbacks), avoidance (avoiding reminders: people, places, activities), negative cognitions and mood (ongoing negative feelings, detachment and alterations in memory), and arousal (including recklessness, irritability, suspicion, lack of concentration/sleeping). The recurring trauma intrusions (trauma loop) described in the APA criteria for diagnosis may disrupt the current moment (living in the present moment) that cause the patient's current state to be one that may be described as dysfunctional (agitated, stressed, sad, suspicious).

Olfactory therapy may offer a unique strategy to break negative, obsessive and trauma thought loops. Odors can ignite memories as well as influence mood and behavior. A medical device provided herein may use odors in a relaxation strategy (lavender is relaxing), associative learning strategy to change mood, to recall a memory (e.g., smell of grandad's pipe) or a learned association (e.g., smell of smoke means danger). A medical device provided herein can also provide an adjunctive therapy to medication or cognitive behavior therapy that can bring attention back to the present moment by disrupting the undesirable thought loops.

It is sometimes beneficial to challenge a subject to sample an odor without directly focusing their attention on such an odor. For example, certain odors influence eating behavior and diet quality, and others break negative, obsessive and trauma-related thought loops.

In order to break the thought loop, focus can be directed toward identification of the odor. A medical device provided herein can deliver an odor that is either unfamiliar (culturally or geographically foreign) or familiar yet not easy to identify or innocuously familiar (e.g., pancakes). An odor that is familiar yet not easy to identify might be a blend of two odors that are not typically associated, for example the smell of tangerine tacos. The medical device may be a wearable device or intended to carry as a personal item. A mobile or computer implemented application is also provided herein to accompany the medical device.

Multiple aspects may contribute to the psychotherapeutic value of a device described herein. In some embodiments, the time lapse data of a user's response to an odor queue is indicative of a mental state that is undesirable or uncomfortable. In other cases, time lapse data of a user's response to an odor queue indicates agitation or recurrence of a trauma. In some cases, the time lapse data is used by an algorithm to refine a trauma healing protocol or method using volatile odorous compounds.

In some cases, the device provided herein prompts a user with particular questions such as listing affiliation of a smell with a particular memory or place. In some cases, the therapeutic device prompts a user with particular questions about mood, mental state, or cognitive ability. In some cases, the device prompts questions about previous memories associated with a set of volatile odorous compounds. In some embodiments, a series of odors is provided to a user such as burning wood, smoke, coffee, tobacco, freshly cut grass, and so forth. In some cases, the series of odors comprises one or more triggering odors or calming odors. Triggering odors refer to odors which are affiliated with negative emotional states, whereas calming odors refer to odors which are affiliated with therapeutic value. In some cases, the odor is a distracting odor requiring mental energy to assess. In some cases, the user may advance to newer regimes of odor assessment as the mental states and outcomes of a user improve, e.g., a reduction in stress and/or a decrease in negative thoughts.

Currently, obesity and eating disorders lack adequate treatments or therapies, and are associated with severe physical and mental health consequences. Odors can take a direct route 1o the limbic system, which includes the amygdala and hippocampus, key regions involved in memory processing and emotion. Accordingly, food odors can be powerfully rewarding cues and can act as a stimulus capable of enhancing appetite or promoting satiety for specific foods. Available evidence indicates that short and often unconscious exposure to food odors triggers sensory-specific appetite (SSA), a phenomenon where appetite for a specific food increases and people choose to consume food items that match the specific food (e.g., sweet vs. savory foods). However, a prolonged (e.g., a 20-min exposure or shorter exposures over 5 consecutive days) and conscious exposure to food odors triggers olfactory-specific satiety (OSS), a phenomenon where pleasantness for the matching food decreases, and hence the intake of foods of similar flavor profile (e.g., sweet) diminishes (see, for example, Boesveldt et al. Detecting Fat Content of Food from a Distance: Olfactory-Based Fat Discrimination in Humans. PLoS ONE. 2014; 9(1):e85977, which is herein incorporated by reference in its entirety). This suggests that in some cases, one or more odors can be provided to a user, wherein the one or more odors enhances appetite or promotes satiety in the user. In some cases, olfactory strategies can be included in behavioral programs to promote weight gain. In some cases, olfactory strategies can be included in behavioral programs to promote weight loss.

Many people suffer from conditions such as anosmia, hyposmia, or parosmia, who experience a partial, or even complete, degradation in olfactory ability. Further, head injuries, neurodegenerative disorders, post-traumatic stress disorder, depression, or diseases such as respiratory viral infections can temporarily or permanently affect a subject's sense of smell. Despite how common sudden onset of smell loss is in people with such diseases or disorders, sense of smell is rarely evaluated or treated in routine medical care, which can have significant negative clinical implication. This may be in part due to lack of simple, reliable smell presentation tools.

The failure to see the mainstream clinical potential of presenting smells may be due to theoretical and practical factors. There is a lack of routine and rapid smell presentation measures available in the art. However, for anosmia, hyposmia, and parosmia, such smell presentation may help diagnose and improve an individual's sense of smell. For subjects desiring to tune their sense of smell to particular odor profiles (i.e., wine, marijuana, olive oil) smell presentation may help improve their sensitivities to certain smells. For those suffering from PTSD, depression, and other emotional states prescribed smell presentations can be used as a therapeutic adjunct. In addition, for those suffering from obesity and/or eating disorders prescribed smell presentations can be used as a therapeutic adjunct. For example, a subject may be experiencing appetite loss or poor appetite as a side effect from cancer and/or its treatment and may use smell presentations as a therapeutic adjunct to increase appetite and/or promote weight gain. In another example, a subject may use smell presentations (such as those described in Example 11) as a therapeutic adjunct to decrease appetite and/or support weight loss.

Recognized herein is a need fora smell presentation program (e.g., systems or methods) that are disposable, portable, and self-administrable to provide a robust, rapid and reliable presentation of odors. The systems and methods described herein may be used to address this need.

### Smell Presentation and Assessment

Disclosed herein are smell presentation programs that include devices, systems, kits, or methods. The smell presentation program may include a source of odors. An electronic system may be included for a user to provide guidance on his or her participation in the smell presentation program. An electronic system may further be included for a user to provide feedback on his or her participation in the smell presentation program. This feedback may be an integral part of the program that reinforces or enhances the smell presentation. An electronic system may further be included to assess the user's cognitive skills by answering questions about the smells or assessing the speed in performing tasks. A smell presentation method may use a device, system, or kit disclosed herein.

In some embodiments, the smell presentation system presents odors to an individual in order to actively induce a specific behavior such as a behavior related to eating, attention, or focus, or to passively influence a measure of neurological function, depression, or quality of life. For example, the smell presentation system can present odors to an individual in order to decrease or increase appetite.

A smell presentation program may be used in conjunction with smell testing. For example, a smell presentation kit may be used to improve a person's sense of smell, and the sense of smell may be assessed along the way. Additionally, similar questions may be asked to a user during the smell presentation and smell assessment method, and the user may respond by providing input to a device that determines the subject's smell capabilities or keeps track of his or her progress.

### Olfactory Training Kits

Disclosed herein, are smell presentation kits (e.g., odor kits). A smell presentation kit may also be referred to as an olfactory training kit. The kit may be included in a system or method of smell presentation. The kit may include one or more smell presentation devices.

A smell presentation kit may take less than 2 minutes to use, and may include training or measuring odor discrimination, intensity and identification based on one odorant. An example includes a flower odor (e.g., Givaudan, perfume compound with main component 2-phenylethanol, CAS-No. 60-12-8). The kit may use odors that are highly familiar to the US population as indicated by published data from existing databases.

A smell presentation kit may include odor patches created with a peel and smell sticker glued to a card via an odorless adhesive. This technology may prevent cross-contamination of odor samples on the same card (e.g., useful for an accurate odor detection in smell presentation or smell testing), may provide standardization of odor delivery across cards and odors (e.g., useful for training or accurately determining odor intensity perception by a user), and may limit residual odor in the air after the test (where residual odor in the air could compromise the ability to identify odors or detect faint odors in the smell presentation kit).

A smell presentation device may include a smell card. The smell card may have multiple smells on it, each covered by a peelable layer and adhered to the card by an adhesive that includes a volatile odorous compound. The smell card may include one or more odor patches adhered to a backing. The one or more odor patches may each include an outward surface (e.g., a flat plastic surface) adhered by an adhesive to another surface or backing. For example, the adhesive may be directly adhered to another surface (e.g., another flat plastic surface) that is directly adhered to a panel or backing. In some aspects, the adhesive is directly adhered to the panel or backing. Each of the odor patches may comprise a distinct odorous volatile compound. The odor patches may also be referred to as, or include, an odor sticker or smell sticker. The odor patches may include plastic, rubber, silicone, paper, or another material. The adhesive may include a glue, a resin, a rubber adhesive, an acrylic adhesive, or any other adhesive capable of retaining an odorous compound for use in the smell presentation. The panel or backing may include paper, card stock, plastic, rubber, metal, silicone, or another material. This kit may be used to retain the odor within the adhesive of one of the odor patches for an extended period of time, while releasing the odor to a user in a uniform, predictable way when the odor label is peeled away from the panel or backing. Each of the odor patches may include a transparent cover. The transparent cover may be lifted by a user to reveal the odor patch.

The smell presentation kit may also include an indicator (such as an image or label) located adjacent to or on each of the odor patches. Images may be adhered to the panel or backing in a similar fashion as the odor patches. The images may indicate to a user the source of the odorous volatile compound. The smell presentation kit may also include a label located adjacent to each of the images. The labels may be adhered to the panel or backing in a similar fashion as the odor patches. The label may comprise the odorous volatile compound's identity. The label may be covered by an opaque cover. The opaque cover may be lifted by a user to reveal the label and therefore reveal the odor's identity. An indicator may also include an odor's intensity, for example, how strong the odor is.

In some aspects, a kit including the one or more odor patches, images, and labels bound either directly or indirectly to a backing is included in a simple system or method of smell presentation that includes, for example, three queries in any order to improve a user's sense of smell. The three queries may identify whether the user detects the odorous compound, whether the user identifies the odor of the odorous compound, and a rating on an intensity by which the user perceives the odor (if the user perceives the odor at all). The kit along with the three queries may aid in smell presentation without the need for a multitude of smells or other complexities.

Disclosed herein, in some cases, are odor kits for smell presentation. The odor kit may include one or more smell cards. The odor kit may be a multifunctional smell presentation kit. The smell presentation kit may comprise at least two surfaces (e.g., 3, 4, or more surfaces) each adhered to a panel by a releasable matrix (e.g., adhesive) that contains an odorous volatile compound (e.g., each having a distinct, separate odor). The first surface adhered by a first releasable matrix comprising an odorous volatile compound may be referred to as an odor sticker or an odor patch. The odor patch may contain an odor released when the two surfaces are separated. In some cases, each of the surfaces is adhered using an adhesive with a different odorous volatile compound. Some embodiments include a first and second releasable matrix. The smell presentation kit may comprise 1, 2, 3, 4, 5, 6, 7, 8, 9, 10, or more smell stickers. The smell presentation kit may comprise 1, 2, 3, 4, 5, 6, 7, 8, 9, 10, or more odorous volatile compounds.

The smell stickers in one kit may be designed to have a stronger smell than smell stickers in another kit. For example, one smell kit may contain smell stickers that would be perceived by a user at 90%, 80%, 70%, 60%, 50%, 40%, 30%, 20%, 10% or less, relative to another smell sticker. The perception may be determined by asking the user to provide a rating. The smell kit with less intense smells may be used sequentially, later than the other kit, in order to keep improving the subject's sense of smell after the subject becomes habituated to the stronger odor kit. Multiple odor kits of descending odor intensities may be included, and used by a subject in a smell presentation regimen.

The surfaces adhered to a panel may comprise plastic, rubber, silicone, or paper. The releasable matrices may be or include an adhesive. The releasable matrices may comprise a glue, a resin, a rubber adhesive, or an acrylic adhesive. A releasable matrix may comprise an oil comprising the odorous volatile compound. Various odors may be used. For example, a familiar household odor may be used. Non-limiting examples include rose, popcorn, coffee, orange, coconut, strawberry, banana, or chocolate odor. Other non-limiting examples include butter, fruit, lemon, soap, eucalyptus, gas, cloves, cinnamon, thyme, lavender, or honey. Some odors may include particular odors that food experts may wish to train in, such as the odors of various varieties of wine, cheese, chemicals, or food ingredients.

A kit may include one or several panels, each including a smell sticker, or each including multiple smell stickers. The separate panels may comprise distinct odors. For example, one panel may include a smell sticker having one odor, and another panel may include another smell sticker having another, different odor.

In some cases, a plurality of odor patches are used per smell presentation kit or per panel. For example, a backing may include a plurality of odor patches comprising a plurality of odorous compounds. A combination of multiple queries enables the use of the training kit to improve a user's sense of smell.

The odor may be selected from a number of odors. For example, a group of smell presentation kits or panels may be manufactured that comprise any of 1, 2, 3, 4, 5, 6, 7, 8, 9, 10, 11, 12, 13, 14, 15, or more smells, wherein each smell presentation includes a plurality of smells. This may allow training a subject on a variety of different odors. A system or method that uses the smell presentation kit may include multiple queries that provide increased discrimination power that may allow for use of a limited number of smells within the group of smell presentation kits. A group of smell presentation kits may be manufactured that comprises no more than 1, 2, 3, 4, 5, 6, 7, 8, 9, 10, 11, 12, 13, 14, or 15, smells, wherein each smell presentation kit includes a plurality of smells. For example, some smell presentation kits may include no more than 12 or 15 total smells, whereas existing smell presentation kits may need a greater number of possible smells within a group of kits to work.

In some cases, the odor (e.g., odorous volatile compound) can comprise the odor of a healthy food item. A healthy food item can refer to foods that provide essential nutrients, such as vitamins, minerals, antioxidants, and dietary fiber, and promote good health. For example, fruits and vegetables (such as, but not limited to apples, oranges, berries, spinach, broccoli, carrots, and bell peppers) can be healthy food items. In some cases, the odor (e.g., odorous volatile compound) can comprise the odor of an indulging food item. Indulging food items can refer to foods that are considered treats or occasional indulgences due to their higher calorie, sugar, or fat content. Indulging food items may not offer significant nutritional benefits. For example, desserts, sweets, and processed snacks (such as, but not limited to cakes, cookies, pastries, ice cream, chocolate bars, chips, crackers, popcorn, and candies) can be indulging food items.

In some cases, the odor (e.g., odorous volatile compound) ranges in concentration. In some embodiments, the odor has a concentration of about from 0.1 mg/mL to 50 mg/mL, 0.1 mg/mL to 20 mg/mL, 0.1 mg/mL to 10 mg/mL, 0.5 mg/mL to 10 mg/mL, 0.5 mg/mL to 5 mg/mL, 0.5 mg/mL to 2 mg/mL, or 0.75 mg/mL to 1.25 mg/mL. In some embodiments, the odor has a concentration of about at least 0.1 mg/mL, 0.25 mg/mL, 0.5 mg/mL, 0.75 mg/mL, 1 mg/mL, 1.25 mg/mL, 1.5 mg/mL, 2 mg/mL, 2.5 mg/mL, 3 mg/mL, 4 mg/mL, or 5 mg/mL. In some embodiments, the odor has a concentration of about at most 0.5 mg/mL, 0.75 mg/mL, 1 mg/mL, 1.25 mg/mL, 1.5 mg/mL, 2 mg/mL, 2.5 mg/mL, 3 mg/mL, 4 mg/mL, 5 mg/mL, 10 mg/mL, 20 mg/mL, or 50 mg/mL.

Where multiple panels are used, each of the individual panels may be designed to be smelled on a different day, such as a different day of the week, or may be smelled at various times during the day. In some embodiments, each of the individual panels is designed to be smelled at different times of days. In some embodiments, each panel is designed to be smelled at intervals of at least 1, 2, 3, 4, 5, 6, 7, 8, 9, 10, 11, 12, 13 or 14 days. In some embodiments, each panel is designed to be smelled with a short exposure time. In some cases, the short exposure time can be at least 1, 2, 3, 4, 5, 6, 7, 8, 9, or 10 minutes in length. In some embodiments, each panel is designed to be smelled with a prolonged exposure time, such as a 20-min exposure or shorter exposures over 5 consecutive days. In some cases, the prolonged exposure time can be at least 11, 12, 13, 14, 15, 16, 17, 18, 19, 20, 21, 22, 23, 24, or 25 minutes in length. In some cases, the prolonged exposure time can be at least 1, 2, 3, 4, 5, 6, 7, 8, 9, or 10 minutes in length over at least 3, 4, 5, 6, 7, 8, 9, or 10 consecutive days.

A smell presentation kit may comprise a series of odor patches adhered to a panel to provide a support structure. The panel may comprise paper, card stock, plastic, rubber, metal, or silicone. The odor patch may adhere the support structure while retaining the odorous volatile compound. In some cases, an intensity of the odor of the volatile compound does not diminish while the odor label adheres the first surface to the support structure for a period of time. In some cases, the intensity of the odor of the volatile compound does not significantly diminish when the first releasable matrix re-adheres the first surface to the panel after the first surface is being peeled away from the support structure and re-adhered to the support structure. The panel may comprise at least 1, 2, 3, 4, 5, 6, 7, 8, 9, or 10 surface areas configured to adhere a surface and/or a releasable matrix. The odor kit may comprise a peeling layer disposed on top of a surface area of the panel. In some cases, at least a portion of the peeling layer is removably coupled to the panel (or support structure). For example, the peeling layer may comprise a transparent cover. Uncoupling a portion of a peeling layer from the panel (or support structure) may release a volatile compound detectable through olfaction. The peel and smell technology described herein is useful because it keeps the odorous compound fresh. This way the compound or odor of the compound does not diminish quickly over time, and the smell presentation can be used multiple times, or the odor may be delivered consistently. In some cases, the smell presentation may be re-used 1, 2, 3, 4, 5, 6, 7, 8, 9, 10, 15, 20, or 25 times, or more, or a range of times defined by any two of the aforementioned integers. The consistency of odor delivery allows for the use of a meaningful rating of or query on an intensity of the odor. The panel or support structure may be a backing such as a paper backing.

In some embodiments, an odor patch comprises or consists of a single surface and the releasable adhesive. The odor patch may be adhered to a support panel by means of the adhesive. The releasable adhesive may include the odorous volatile compound.

In some cases, the kit includes one or more odor patches. The odor patches may be indirectly adhered to the panel. For example, one or more odor patches may be adhered to one or more other surfaces (e.g., plastic surfaces) adhered to a panel or backing. Some such embodiments include a first surface (e.g., odor patch) adhered by an adhesive with an odorous compound to another surface that is directly adhered to the panel or backing. Some embodiments include a second and third surface (e.g., second and third odor labels, in any order) each adhered by an adhesive without the odorous compound to other surfaces that are directly adhered to the panel or backing. The panel may include a symbol or code, such as a URL or QRL code that links to a component of a smell presentation system, such as for entering or obtaining information related to a smell presentation program.

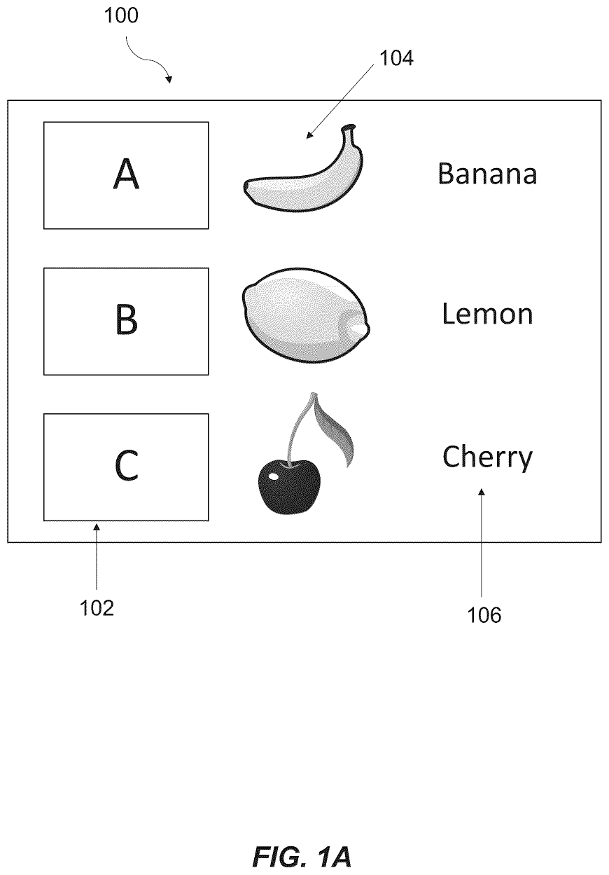

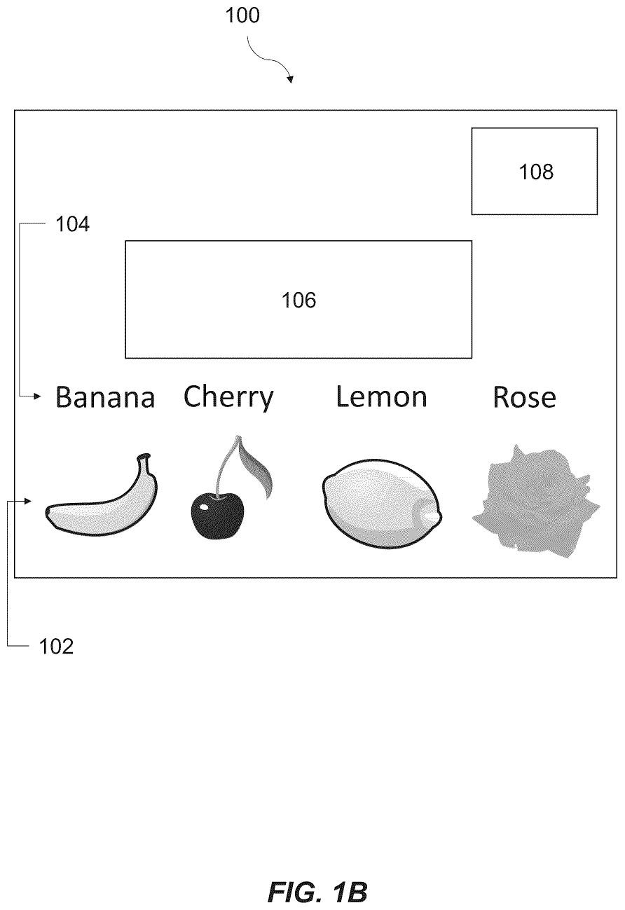

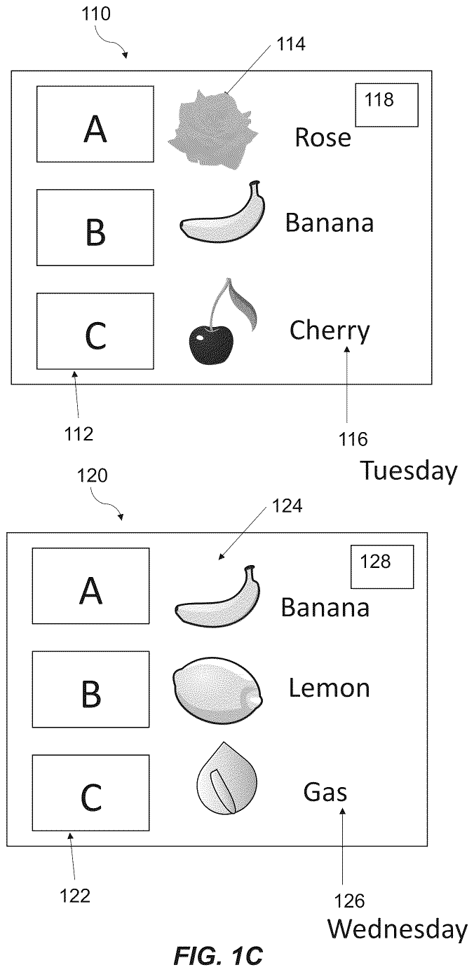

An example of a smell presentation device is depicted in FIG. 1A, which shows a panel 100 with a first column 102 comprising a plurality of patches. Each of the plurality of patches may comprise a distinct odorous volatile compound. As depicted in FIG. 1A, the panel 100 comprises three patches. In other embodiments, the panel 100 may comprise 2, 4, 5, 6, 7, 8, 9, 10, 11, 12, 13, 14, 15, 16, 17, 18, 19, or 20 or more patches. The panel 100 may comprise a second column 104 comprising an image located adjacent to each of the plurality of patches. The images may indicate to a user the source of the odorous volatile compound. As illustrated in FIG. 1A, the panel 100 may comprise three images comprising a banana, a lemon, and a cherry. In other embodiments, the panel 100 may comprise 2, 4, 5, 6, 7, 8, 9, 10, 11, 12, 13, 14, 15, 16, 17, 18, 19, or 20 or more images. The panel 100 may also comprise a third column 106 comprising a label located adjacent to each image. The label may comprise the odorous volatile compound's identity. In one embodiments, as shown in FIG. 1A, the labels comprise “Banana” located adjacent to the banana image, “Lemon” located adjacent to the lemon image, and “Cherry” located adjacent to the cherry image. In other embodiments, the panel 100 may comprise 2, 4, 5, 6, 7, 8, 9, 10, 11, 12, 13, 14, 15, 16, 17, 18, 19, or 20 or more labels. The panel 100 may also comprise a transparent cover for each of the plurality of patches in the first column 102. The transparent cover can be releasably coupled to the panel 100 and be lifted by a user to access the odorous volatile compound. The transparent cover may retain the odor of the odorous volatile compound for extended use. The panel 100 may also comprise an opaque cover for each of the labels in the third column 106. The opaque cover may hide the identity of the odorous volatile compounds in the first column 102. The opaque cover can be releasably coupled to the panel 100 and be lifted by a user to reveal the label.

Another example of a smell presentation device is depicted in FIG. 1B. FIG. 1B shows a panel 100 with a first row 102 comprising a plurality of patches. Each of the plurality of patches may comprise a distinct odorous volatile compound. In the embodiment depicted in FIG. 1A, the panel 100 comprises four patches. In other embodiments, the panel 100 may comprise 2, 4, 5, 6, 7, 8, 9, 10, 11, 12, 13, 14, 15, 16, 17, 18, 19, or 20 or more patches. Each of the patches in FIG. 1B may further comprise an image located on the patch. The image indicates to a user the source of the odorous volatile compound. The panel 100 may comprises a second row 104 comprising a label located adjacent to each of the patches/images. In the embodiment depicted in FIG. 1A, the panel 100 comprises four labels. The labels comprise “Banana” located adjacent to the patch/image of the banana, “Cherry” located adjacent to the patch/image of the cherry, “Lemon” located adjacent to the patch/image of the lemon, and “Rose” located adjacent to the patch/image of the rose. In other embodiments, the panel 100 may comprise 2, 4, 5, 6, 7, 8, 9, 10, 11, 12, 13, 14, 15, 16, 17, 18, 19, or 20 or more labels. The panel 100 may also comprise a transparent cover for each of the plurality of patches in the first row 102. The transparent cover can be releasably coupled to the panel 100 and be lifted by a user to access the odorous volatile compound. The transparent cover may retain the odor of the odorous volatile compound for extended use. The panel 100 may also comprise an opaque cover for each of the labels in the second row 104. The opaque cover may hide the identity of the odorous volatile compounds in the first row 102. The opaque cover can be releasably coupled to the panel 100 and be lifted by a user to reveal the label. The panel 100 may comprise a text box 106 that may contain instructions for a user on how to complete the smell presentation. The panel 100 may comprise a QR code 108 that a user may scan with an appropriate device. The QR code can direct a user to a mobile phone application or website where the user may receive instructions on completing the smell presentation. The phone application or website may also allow a user to input information after completing the smell presentation.

A smell presentation kit comprises at least 1, 2, 3, 4, 5, 6, 7, 8, 9, 10, 11, 12, 13, 14, 15, 16, 17, 18, 19, 20 or more panels or smell presentation devices. In some embodiments, each panel is designated with a specific day of the week. An example of a smell presentation kit comprising multiple panels is depicted in FIG. 1C. FIG. 1C depicts a first panel 110 and a second panel 120. In some embodiments, the first panel 110 has a first column 112 comprising a plurality of patches. Each of the plurality of patches may comprise a distinct odorous volatile compound. In some embodiments, the panel 110 comprises three patches. In other embodiments, the first panel 110 may comprise 2, 4, 5, 6, 7, 8, 9, 10, 11, 12, 13, 14, 15, 16, 17, 18, 19, or 20 or more patches. The first panel 110 may comprise a second column 114 comprising an image located adjacent to each of the plurality of patches. The images may indicate to a user the source of the odorous volatile compound. The panel 110 may comprise three images comprising a rose, a lemon, and a cherry. In other embodiments, the panel 110 may comprise 2, 4, 5, 6, 7, 8, 9, 10, 11, 12, 13, 14, 15, 16, 17, 18, 19, or 20 or more images. The panel 110 may also comprise a third column 116 comprising a label located adjacent to each image. The label may comprise the odorous volatile compound's identity. In some embodiments, the labels comprise “Rose” located adjacent to the banana image, “Banana” located adjacent to the lemon image, and “Cherry” located adjacent to the cherry image. In other embodiments, the panel 110 may comprise 2, 4, 5, 6, 7, 8, 9, 10, 11, 12, 13, 14, 15, 16, 17, 18, 19, or 20 or more labels. The panel 110 may also comprise a transparent cover for each of the plurality of patches in the first column 112. The transparent cover can be releasably coupled to the panel 110 and be lifted by a user to access the odorous volatile compound. The transparent cover may retain the odor of the odorous volatile compound for extended use. The panel 110 may also comprise an opaque cover for each of the labels in the third column 116. The opaque cover may hide the identity of the odorous volatile compounds in the first column 112. The opaque cover can be releasably coupled to the panel 110 and be lifted by a user to reveal the label.

The second panel 120 may have a first column 122 comprising a plurality of patches. Each of the plurality of patches may comprise a distinct odorous volatile compound. The panel 120 may comprise three patches. The second panel 120 may comprise 2, 4, 5, 6, 7, 8, 9, 10, 11, 12, 13, 14, 15, 16, 17, 18, 19, or 20 or more patches. The second panel 120 may comprise a second column 124 comprising an image located adjacent to each of the plurality of patches. The images may indicate to a user the source of the odorous volatile compound. The panel 120 may comprise three images comprising a banana, a lemon, and a gas (flame). The panel 120 may comprise 2, 4, 5, 6, 7, 8, 9, 10, 11, 12, 13, 14, 15, 16, 17, 18, 19, or 20 or more images. The panel 120 may also comprise a third column 126 comprising a label located adjacent to each image. The label may comprise the odorous volatile compound's identity. In one embodiment, the labels comprise “Banana” located adjacent to the banana image, “Lemon” located adjacent to the lemon image, and “Gas” located adjacent to the gas (flame) image. In other embodiments, the panel 120 may comprise 2, 4, 5, 6, 7, 8, 9, 10, 11, 12, 13, 14, 15, 16, 17, 18, 19, or 20 or more labels. The panel 120 may also comprise a transparent cover for each of the plurality of patches in the first column 122. The transparent cover can be releasably coupled to the panel 120 and be lifted by a user to access the odorous volatile compound. The transparent cover may retain the odor of the odorous volatile compound for extended use. The panel 120 may also comprise an opaque cover for each of the labels in the third column 126. The opaque cover may hide the identity of the odorous volatile compounds in the first column 122. The opaque cover can be releasably coupled to the panel 110 and be lifted by a user to reveal the label.

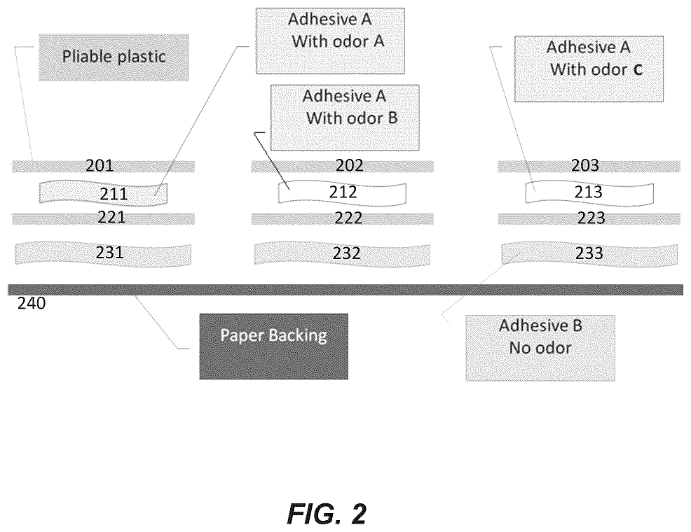

Any of the components shown in FIG. 2 may be used. The kit may include a first surface 201 adhered by an adhesive 211 comprising an odorous compound (e.g., odor patch 1). The kit may include a second surface 202 adhered by an adhesive 212 comprising a different odorous compound(e.g., odor patch 2) to the first odor patch. The kit may include a third surface 203 adhered by an adhesive 213 comprising another different odorous compound (e.g., odor patch 3) to both the first and second odor patches. Odor patches 1-3 may all include the same adhesive but for the odorous compound. The adhesive 211 of odor patch 1 may be further adhered to a fourth surface 221. The adhesive 212 of odor patch 2 may be further adhered to a fifth surface 222. The adhesive 213 of odor patch 3 may be further adhered to a sixth surface 223. The fourth surface 221 may be adhered by an adhesive 231 to a panel 240. The fifth surface 222 may be adhered by an adhesive 232 to the panel 240. The sixth surface 223 may be adhered by an adhesive 233 to the panel 240. The adhesives adhering the fourth, fifth, or sixth surfaces to the panel may all be the same type of adhesive as each other. The adhesives adhering the fourth, fifth, and/or sixth surfaces to the panel may all be the same type of adhesive as the adhesives of the first, second, and/or third odor labels but for the odorous compound. In some alternatives, the first, second, and third odor patches are all directly adhered to a single surface that is adhered to the panel, whereas in other cases the first, second, and third odor patches are all directly adhered to separate surfaces that are adhered to the panel. The terms first, second, third, fourth, fifth, and sixth are used for convention, and do not necessarily denote a specific order. For example, the first, second, and third odor labels may be in any order, or in any placement on the panel.

### Olfactory Training Systems

An odor kit may be part of a smell presentation system. The system may comprise a means for registering if the odorous volatile compound is detected by a user. The registering may comprise a first query for obtaining a first input on whether the user detects, by olfaction, the odorous volatile compound. The first input on whether the user detects, by olfaction, the odorous volatile compound may comprise a determination by the user if they detected the odorous volatile compound of each of the odor patches on the panel. A user can consider any of the odor labels on the panel to attempt to detect, by olfaction, the odorous volatile compound of that surface. The user may consider a first surface or a second surface each comprising an odor of a volatile compound. The registering may comprise a second query for obtaining a second input on whether the user identifies the source of the odorous volatile compound. The registering may comprise a third query for obtaining a third input of a rating of on an intensity of the odor as perceived by the user. The third query may comprise a questionnaire associated with a rating of an intensity of the odor. The third input on the intensity of the odor as perceived by the user, may comprise a quantitation of the intensity of the odor. The queries need not occur in the order presented herein. For example, the second or third query may be presented to the user first, or the first or second query may be presented to the user last.

A system or method may include providing or receiving a rating, such as from a user of a smell kit or smell test. The rating may include a numerical rating. The rating may include an alphanumeric rating. The rating may be on a scale. The scale may include a psychometric response scale. The scale may include a continuous scale. The scale may include a Likert scale. The scale may include an ordinal scale. The scale may include discrete categories. Examples of scales include 0 to 100, 1 to 100, 10 to 10, 1 to 10, or 1 to 5.

The smell presentation system may further comprise additional queries for obtaining additional input from a user. The additional queries may comprise a query for obtaining an input from a user on the user's physical health. The additional queries may comprise a query for obtaining an input from a user on the user's mood. The additional queries may comprise a query for obtaining an input from a user on the user's mental health status. The additional queries may comprise a query for obtaining an input from a user on the user's cognitive health status. The additional queries may comprise a query about the user's memory of the odor or prior experience with the odor. The additional queries may comprise a query for obtaining an input comprising any combination of the user's physical health, mood, cognitive or mental health status.

Disclosed herein are systems for smell presentation. The system may comprise a smell presentation kit, or an odor kit. The odor kit may comprise a support structure comprising a first surface area. The odor kit may further comprise a first peeling layer disposed on top of the first surface area. At least a portion of the first peeling layer may be removably coupled to the support structure. The odor kit may further comprise a chemical substance disposed between the first peeling layer and first surface area. Uncoupling the portion of the first peeling layer from the support structure may release a volatile compound. The volatile compound may be detectable through olfaction. An example of an odor kit includes a support with multiple smell stickers on it. The smell stickers may separately include distinct odors, and may be adhered to the support. The odor kit may be similar to an olfactory assessment kit disclosed herein, but may have multiple smell stickers instead of having one or more blank stickers without smell. Peeling layers and a support structure are used to describe some odor kits, but an odor kit may entail odor labels adhered to a backing or other aspects herein described.

The system may include a module(e.g., a first module) for receiving a first input from a user. The first input may correlate whether the user detected the volatile compound through olfaction upon uncoupling the portion of the first peeling layer. The first module may receive the first input through a user interface. The system may include a module (e.g., a second module) for receiving a second input from a user. The second input may correlate whether the user identified a type of odor corresponding to the volatile compound upon uncoupling the portion of the first peeling layer. The second module may receive the second input through the user interface. The system may include a module (e.g., a third module) for receiving a third input from a user. The third input may correlate to a rating of an intensity level provided by the user for the type of odor. The third module may receive the third input through the user interface. The system may include a module (e.g., a fourth module) for determining an olfaction capability by the user based on the first input, the second input, and the third input. The fourth module may generate an output based on the determined olfaction capability. The modules may be separate or combined, or may be performed or used in any order.

The system may comprise a computer-implemented system. The computer-implemented system may comprise a first module for receiving a first input from a user. The first input may correlate whether the user detected the volatile compound through olfaction upon uncoupling the portion of the first peeling layer. The first module may receive the first input through a user interface. The computer-implemented system may further comprise a second module for receiving a second input from a user. The second input may correlate whether the user identified a type of odor corresponding to the volatile compound upon uncoupling the portion of the first peeling layer. The second module may receive the second input through the user interface. The computer-implemented system may further comprise a third module for receiving a third input from a user. The third input may correlate to a rating of an intensity level provided by the user for the type of odor. The third module may receive the third input through the user interface. The computer-implemented system may further comprise a fourth module for determining an olfaction capability by the user based on the first input, the second input, and the third input. The fourth module can generate an output based on the determined olfaction capability. The system may include a measurement of the time it takes the user to respond to a prompt.

Also disclosed herein, in some cases, are smell presentation systems. The system may comprise a panel comprising a first peelable surface adhered to the panel by a first adhesive matrix. The adhesive matrix may comprise an adhesive. The adhesive matrix may comprise a volatile compound configured to be detected by a person of ordinary olfactory sensitivity, and configured to be undetectable by a person having an impaired olfactory sensitivity. The first adhesive matrix may be an adhesive comprising the volatile compound. The compound may comprise an odor identifiable by the person of ordinary olfactory sensitivity and unidentifiable by the person having the impaired olfactory sensitivity. The system may further comprise a symbol or code identifiable by a computational device. The symbol or code may be configured to obtain a first input on whether the volatile compound is detected in the first adhesive matrix, a second input on whether the odor is identified, and a third input on a perceived rating of an intensity of the odor.

The smell presentation system may include a network over which inputs are transmitted. The inputs may be provided by a user or a medical practitioner. The smell presentation system may include a network over which the first input is transmitted. The smell presentation system may further comprise a network over which the second input is transmitted. The smell presentation system may further comprise a network over which a third input is transmitted. The smell presentation system may further comprise a network over which any of the additional inputs is transmitted. The inputs may be obtained or transmitted in any order, or may include only some of the inputs described herein.

A user with a compromised sense of smell may be unable to detect, by olfaction, the odorous volatile compound of the odor patches. The user with a compromised sense of smell may be unable to is unable to identify the odor. The user with a compromised sense of smell may be unable to perceives the odor below a designated intensity. The designated intensity may be at about 20 on a scale of 0-100, or may be at about 2 on a scale of 0-10. The designated intensity may be at least about 20 on a scale of 0-100, or may be at least about 2 on a scale of 0-10.

The query may be delivered orally, in written form, or by a computational device or telephone (e.g., smart phone). The system for registering if the odorous volatile compound is detected by a user may comprise a symbol or code. The symbol or code may be printed on the panel. The symbol or code may comprise a quick response (QR) code or a web address. The symbol or code may be indicative of the composition of the odor patches. The symbol or code may be indicative that a first releasable matrix releases a first smell. The symbol or code may indicate a smell that a surface of a panel releases. The system may comprise a computer or an operator that may be configured to read the symbol or code to generate an output of a query.

The system may comprise a first output comprising the first query. The system may comprise a second output comprising the second query. The system may comprise a third output comprising the third query. The system may comprise additional outputs comprising any additional queries. The output may be a computer generated output.

The smell presentation system may include a network over which outputs (e.g., queries) are transmitted. The smell presentation system may comprise a network over which the first output is transmitted. The smell presentation system may further comprise a network over which the second output is transmitted. The smell presentation system may further comprise a network over which a third output is transmitted. The smell presentation system may further comprise a network over which any additional outputs are transmitted. The outputs may be transmitted in any order, or only some of the outputs may be transmitted.

The smell presentation system may further comprise a computational device comprising an output device configured to indicate an output. The output may indicate that the user's sense of smell may or may not have improved when the first input indicates that the odorous volatile compound is detected by the user. The output may indicate that the user's sense of smell may or may not have improved when the second input indicates that the odor is identified by the user. The output may indicate that the user's sense of smell may or may not have improved when the third input indicates that the intensity of the odor is perceived by the user at or above the threshold intensity. The output may indicate that the user's sense of smell may or may not have improved when the first input indicates that the odorous volatile compound is detected by the user, the second input indicates that the odor is identified by the user, and the third input indicates that the intensity of the odor is perceived by the user at or above the threshold intensity. The computational device may comprise a telephone (e.g., smart phone) or tablet.

The smell presentation system may further comprise a computational device comprising an output device, as described above, which is also configured to present the data (e.g., output) to a peer group. A peer group can refer to a collection of individuals within the same field or specialty who share similar levels of expertise, knowledge, and/or professional experiences. In some cases, the peer group can comprise a medical profession. In some cases, a peer group can comprise a group of medical professionals. For example, the peer group can be a group of medical professionals who can seek advice, share experiences, and discuss clinical challenges relating to the smell presentation system. In some cases, a peer group of medical professionals can play a role in improving patient (e.g., a subject and/or user) care and outcomes.

Providing the input may help a user reinforce his or her sense of smell. Likewise, providing the input may improve a user's sense of smell, for example, by engaging areas of the user's involved in smell cognition to a greater extent than not providing the input would. For example, by considering and inputting information into an input device related to the user's ability to detect an odorous volatile substance, identify an odor of the odorous volatile substance, and to quantify or rate the intensity of the odor, the user may improve his or her sense of smell. The improvement may be to a greater extent than in a similar case without the use of one of the three queries, or without the use of any of the queries. Additionally, observing an image of the odor on the panel while smelling the odor may engage the brain further, and further improve the smell presentation program. Thus, the subject or user's treatment of his or her sense of smell is improved beyond what it would otherwise be without these aspects. The information provided or inputted by the user may be stored in a computer memory, and accessed by a system component to provide recommendations or indicate the user's progress.

The user may have an infection such as a coronavirus infection, a neurodegenerative disease, a head trauma, a cognitive impairment, anosmia, or another disorder, which results in the compromised sense of smell. The user may have anosmia, hyposmia, or parosmia. The system may detect if the sense of smell of a user with any of the conditions mentioned above has improved.

### Olfactory Training Methods

Disclosed herein are smell presentation methods. The method may be used to train and improve a subject's sense of smell. The method may comprise use of a smell presentation kit. The smell presentation kit may be integrated into a smell presentation system. The smell presentation system may include an electronic system as described herein. The electronic system may be configured to receive a plurality of inputs from the user. In some embodiments, the electronic system is part of or comprises a computer.

The method may be used to train the subject to dismiss thought loops. The method may be used to reduce or increase appetite in the subject. The method may be used to reduce or increase body weight in the subject. The method may include asking the subject questions, having the subject do a self-assessment by considering or addressing questions, or obtaining information from the subject related to the perceived mental state.

In some aspects, provided herein is a method of treating an mental illness comprising administering a smell kit provided herein. In some embodiments, the method comprises administering a smell kit comprising one or more smell cards. In some embodiments, the method comprises administering a smell kit comprising one or more smell tests. In some embodiments, the method comprises administering a smell kit comprising one or more questionnaires. In some embodiments, the method further comprises providing a suggested treatment or behavioral modification. In some embodiments, the method further comprises providing a suggestion to quarantine, self-isolate, or exclude a subject from group activities. In some embodiments, the method further comprises providing an instruction to quarantine to a subject. In some embodiments, the method further comprises providing an instruction to self-isolate to a subject. In some embodiments, the method further comprises providing an instruction to self-isolate a subject.

The subject may suffer from an olfactory impairment. The subject may be a user or patient. The subject may use a smell presentation system or kit disclosed herein. The olfactory impairment may include anosmia, hyposmia, or parosmia. The olfactory impairment may be resultant from a disease, disorder, or injury.

The method may comprise providing a subject with a panel as described herein. The panel may comprise a plurality of patches. Each of the plurality of patches may comprise a distinct odorous volatile compound. The panel may further comprise an image located adjacent to or on each of the plurality of patches. Each image may indicate to a subject the source of the odorous volatile compound. The panel may further comprise a label located adjacent to each image. The label may comprise the odorous volatile compound's identity.

The method may include use of a smell presentation kit may be with olfactory function measurement components that can be objectively assessed to yield a falsification metric and the ability to calculate the probability of passing the test in the absence of smell ability. For example, a smell test odor detection subtest with 3 possibilities may have a guessing probability of 33%. An odor intensity subtest may rely on a subjective experience of the participant and in some instances may not be directly falsified. The odor identification subtest may include two possibilities: a first try, which is a 4-alternative forced choice task with guessing probability of 25% and a second try, which is a 3-alternative forced choice task with guessing probability of 33%.

The method may include asking the subject questions, having the subject do a self-assessment by considering or addressing questions, or obtaining information from the subject related to the questions. An example of a question is whether the subject detects, by olfaction, an odorous volatile compound contained within an adhesive matrix. The subject may be asked to identify what odor is detected. The subject may be asked to rate an intensity of the odor he or she perceives. The subject may be subjected to this series of questions for multiple odorous volatile compounds, each containing different odorous volatile compounds having various odors. For example, this series of questions may be asked in any order to a subject for each smell sticker of a smell presentation kit.

The method may include receiving or integrating a plurality of inputs from a subject. The inputs received may relate to a first question or instruction, a second question or instruction, and a third question or instruction (in any order). Some embodiments include receiving inputs from the subject on whether the subject detects, by olfaction, the odorous volatile compound in an adhesive matrix or matrices. Some embodiments include receiving inputs from the subject on whether the subject identifies the odor of the volatile compound. Some embodiments include receiving inputs from the subject on a rating of an intensity of the odor as perceived by the subject.

The method may be included as part of an overall health assessment of a subject. For example, the method may comprise receiving a plurality of additional inputs from the subject. The additional inputs received may include input about the subject's physical health. The additional inputs received may include input about the subject's mood. The additional inputs received may include input about the subject's mental health status. The additional inputs received may include input on any combination of physical health, mood, and mental health status.

The method may comprise receiving any of the plurality of inputs disclosed herein from a subject a certain number of times per day, and over the course of a certain number of days, weeks, months, or years. For example, the method may comprise receiving any of the plurality of inputs from a subject 1, 2, 3, 4, 5, 6, 7, 8, 9, or 10 or more times per day. The method may further comprise receiving the plurality of inputs from a subject daily for the course of 1, 2, 3, 4, 5, 6, 7, 8, 9, 10, 11, 12, 13, 14, 15, 16, 17, 18, 19, 20, 21, 22, 23, or 24 or more weeks. The method may further comprise receiving the plurality of inputs from a subject daily for the course of 1, 2, 3, 4, 5, 6, 7, 8, 9, 10, 11, or 12 or more months. The method may further comprise receiving the plurality of inputs from a subject daily for the course of 1, 2, 3, 4, or 5 or more years.

The method may comprise instructing a subject to complete a questionnaire in order to receive the plurality of inputs from the subject. The method may instruct a subject to complete the questionnaire a certain number of times per day, and over the course of a certain number of days, weeks, months, or years. For example, the method may instruct a subject to complete the questionnaire twice daily over 8 weeks. The questionnaire may comprise a first question or instruction, a second question or instruction, and a third question or instruction (in any order). The first question or instruction may be designed for determining whether the subject detects, by olfaction, the odorous volatile compound in the first adhesive matrix. The second question or instruction may be designed for determining whether the subject identifies the odor of the volatile compound. The third question or instruction may be designed for determining a rating of an intensity of the odor as perceived by the subject. The third question or instruction may comprise a question or instruction to determine a quantitation of the intensity of the odor as perceived by the subject. The question or instruction to determine a quantitation of the intensity of the odor as perceived by the subject may comprise an image of a slider. The quantitation of the intensity of the odor as perceived by the subject may comprise a scale of 0-10. The quantitation of the intensity of the odor as perceived by the subject may comprise a scale of 1-10. The quantitation of the intensity of the odor as perceived by the subject may comprise a scale of 0-100. The quantitation of the intensity of the odor as perceived by the subject may comprise a scale of 1-100.

The method may comprise storing the plurality of inputs each time the plurality of inputs is received. For example, if the subject is instructed to complete the questionnaire twice daily over 8 weeks, then the method may comprise storing the inputs received from the subject twice daily over the 8 weeks. For example, the method may comprise storing any of the plurality of inputs from a subject 1, 2, 3, 4, 5, 6, 7, 8, 9, or 10 or more times per day. The method may further comprise storing the plurality of inputs from a subject daily for the course of 1, 2, 3, 4, 5, 6, 7, 8, 9, 10, 11, 12, 13, 14, 15, 16, 17, 18, 19, 20, 21, 22, 23, or 24 or more weeks. The method may further comprise storing the plurality of inputs from a subject daily for the course of 1, 2, 3, 4, 5, 6, 7, 8, 9, 10, 11, or 12 or more months. The method may further comprise receiving the storing of inputs from a subject daily for the course of 1, 2, 3, 4, or 5 or more years.

The smell presentation method may further comprise determining, based on the subject's inputs, whether the subject's sense of smell has improved. For example, the subject's sense of smell may be determined to have improved if a number or percentage of odors detected is increased, if a number or percentage of odors identified is increased, or if odor detection intensity ratings are improved or increased, or a combination thereof. The improvement(s) may be made relative to a baseline.

In some embodiments, the smell presentation method may comprise providing a subject a plurality of panels. Each of the plurality of panels may comprise a plurality of patches. Each of the plurality of patches may comprise a distinct odorous volatile compound. The panel may further comprise an image located adjacent to or on each of the plurality of patches. Each image may indicate to a subject the source of the odorous volatile compound. The panel may further comprise a label located adjacent to each image. The label may comprise the odorous volatile compound's identity.

The smell presentation method may further comprise exposing the subject to the odorous volatile compound comprised in each of the plurality of patches of a first panel of the plurality of panels a certain number of times per day over a certain number of days, weeks, months, or years. For example, a subject may be exposed to the odorous volatile compounds of a first panel twice a day over 8 weeks. The method may further comprise exposing the subject to the odorous volatile compound comprised in each of the plurality of patches of a second panel of the plurality of panels a certain number of times per day over a certain number of days, weeks, months, or years. For example, a subject may be exposed to the odorous volatile compounds of a second panel twice a day over 8 weeks. The method may further comprise exposing the subject to the odorous volatile compound comprised in each of the plurality of patches of a third panel of the plurality of panels a certain number of times per day over a certain number of days, weeks, months, or years. For example, a subject may be exposed to the odorous volatile compounds of a third panel twice a day over 8 weeks. The method may further comprise exposing the subject to the odorous volatile compound comprised in each of the plurality of patches of an additional 1, 2, 3, 4, 5, 6, 7, 8, 9, or 10 or more panels.

The method may comprise exposing the subject to the odorous volatile compounds of a panel any other number of times per day, and over the course of any other number of days, weeks, months, or years. For example, the method may comprise exposing the subject to the odorous volatile compounds of a panel 1, 2, 3, 4, 5, 6, 7, 8, 9, or 10 or more times per day. The method may comprise exposing the subject to the odorous volatile compounds of a panel for at least 5, 10, 15, 20, 25, 30, 35, 40, 45, 50, 55, or 60 seconds. The method may comprise exposing the subject to the odorous volatile compounds of a panel for at least 1, 2, 3, 4, 5, 6, 7, 8, 9, 10, or more than 10 minutes. The method may further comprise exposing the subject to the odorous volatile compounds of a panel daily for the course of 1, 2, 3, 4, 5, 6, 7, 8, 9, 10, 11, 12, 13, 14, 15, 16, 17, 18, 19, 20, 21, 22, 23, or 24 or more weeks. The method may further comprise exposing the subject to the odorous volatile compounds of a panel daily for the course of 1, 2, 3, 4, 5, 6, 7, 8, 9, 10, 11, or 12 or more months. The method may further comprise exposing the subject to the odorous volatile compounds of a panel daily for the course of 1, 2, 3, 4, or 5 or more years. In some embodiments, the method comprises exposing the subject to the odorous volatile compounds of a panel with a short exposure time. In some cases, the short exposure time can be at least 1, 2, 3, 4, 5, 6, 7, 8, 9, or 10 minutes in length. In some embodiments, the method comprises exposing the subject to the odorous volatile compounds of a panel with a prolonged exposure time, such as a 20-min exposure or shorter exposures over 5 consecutive days. In some cases, the prolonged exposure time can be at least 11, 12, 13, 14, 15, 16, 17, 18, 19, 20, 21, 22, 23, 24, or 25 minutes in length. In some cases, the prolonged exposure time can be at least 1, 2, 3, 4, 5, 6, 7, 8, 9, or 10 minutes in length over at least 3, 4, 5, 6, 7, 8, 9, or 10 consecutive days.

The method may comprise, after being exposed to a first panel as described above, assessing the subject's sense of smell. The method may further comprise, after being exposed to a second panel as described above, assessing the subject's sense of smell. The method may further comprise, after being exposed to a third panel as described above, assessing the subject's sense of smell. The method may further comprise, after being exposed to a fourth, fifth, sixth, seventh, eighth, ninth, tenth, eleventh, twelfth, or thirteenth panel as described above, assessing the subject's sense of smell.

The smell presentation method may further comprise assessing the user's mental state before and/or after input on the user's physical health. In some cases, the method comprises assessing the user's mental state before and/or after input on the user's mood. In some embodiments, the method comprises assessing the user's mental state before and/or after input of the user's mental health status. In some cases, the method comprises determining whether the user's mental state has improved compared to a placebo or compared to the user's mental state prior to input on the user's physical health. In some embodiments, the method further comprises, assessing the user's mental state before and/or after exposing the user to the odorous volatile compounds. In some cases, the method further comprises, assessing the user's mental state before and/or after completing a smell presentation test. In some embodiments, the method further comprises, assessing the user's mental state before and/or after the user to completes the smell presentation test over a short exposure time, over a long exposure time, and/or at least twice daily over the course of at least eight weeks. In some cases, the method further comprises, determining whether the user's mental state has improved compared to a placebo or compared to the user's mental state prior to providing the user a panel.

Use of a smell presentation kit may comprise completing a smell test aspect of a smell presentation program, for example, completing a smelling aspect and then recording or inputting answers to various questions into a device. As such, the smell presentation method may entail smelling odorous volatile compounds, and then completing a smell presentation test. The smell presentation test may be completed using a smell presentation device or kit described herein. The smell presentation test may involve various aspects such as determining if a subject detects the odorous volatile compound, determining if the subject can identify the odor, or having the subject provide a rating of his or her perceived intensity of the odor.

The method may include obtaining or providing input on whether the user detects, by olfaction, the odorous volatile compounds of a smell presentation kit described herein. The method may include obtaining or providing input on whether the user identifies the source of the odorous volatile compounds. The method may include obtaining or providing input on a rating on the odor of each odorous volatile compound. The method may further include obtaining or providing input on the user's physical health, mood, or mental health status, or a combination thereof.

In some embodiments, the computer instructs the user to use a smell presentation kit or complete a smell presentation test 1, 2, 3, 4, 5, 6, 7, 8, 9, or 10 or more times per day. In some embodiments, the computer instructs the user to use a smell presentation kit or complete the smell presentation test daily for the course of 1, 2, 3, 4, 5, 6, 7, 8, 9, 10, 11, 12, 13, 14, 15, 16, 17, 18, 19, 20, 21, 22, 23, or 24 or more weeks. In some embodiments, the computer instructs the user to use a smell presentation kit or complete the smell presentation test daily for the course of 1, 2, 3, 4, 5, 6, 7, 8, 9, 10, 11, or 12 or more months. In some embodiments, the computer instructs the user to use a smell presentation kit or complete the smell presentation test daily for the course of 1, 2, 3, 4, or 5 or more years.

In some embodiments, the user self-determines to use a smell presentation kit or complete a smell presentation test 1, 2, 3, 4, 5, 6, 7, 8, 9, or 10 or more times per day. In some embodiments, the user self-determines to use a smell presentation kit or complete the smell presentation test daily for the course of 1, 2, 3, 4, 5, 6, 7, 8, 9, 10, 11, 12, 13, 14, 15, 16, 17, 18, 19, 20, 21, 22, 23, or 24 or more weeks. In some embodiments, the user self-determines to use a smell presentation kit or complete the smell presentation test daily for the course of 1, 2, 3, 4, 5, 6, 7, 8, 9, 10, 11, or 12 or more months. In some embodiments, the user self-determines to use a smell presentation kit or complete the smell presentation test daily for the course of 1, 2, 3, 4, or 5 or more years.

The smell presentation method may include determining the subject has improved his or her olfactory ability or sense of smell. The improvement may be identified when the subject is determined to detect, by olfaction, one or more odorous volatile compounds among a plurality of smell patches, when the subject identifies the odor(s) of the one or more odorous volatile compounds, or when the subject perceives the intensity of the odor(s) at or above a certain threshold, or when a combination of such factors have been met.

The smell presentation method may include determining the subject has not improved his or her olfactory ability or sense of smell. The lack of improvement may be identified when the subject is determined to not detect, by olfaction, one or more odorous volatile compounds among a plurality of smell patches, when the subject does not identify the odor(s) of the one or more odorous volatile compounds, or when the subject perceives the intensity of the odor(s) at or below a certain threshold, or when a combination of such factors have been met.

At least six considerations may be important for large scale deployment of a smell presentation: (a) it may need to be fast and simple to administer without trained personnel, (b) easily identified odorants may need to be chosen, (c) there may need to be enough odorants so people can use a smell presentation kit or take a smell presentation test frequently, (d) the way the odorants are offered to participants may be as uniform as possible, (e) the smell presentation kit or test is designed to avoid physical contamination, and (f) the correct answers may not be not easy to guess. Speed may be important because smell presentation especially for population surveillance may be fast, e.g., for building admittance or when a subject is experiencing thought loops and may be aided by fast access. Odorant choice may be important because the odorants may be familiar within the cultural or geographic context where the smell presentation kit or test is used, to minimize misattributions that do not depend on the ability to smell. Odorants in some cases may not have a pungent component due to trigeminal activation (e.g., mint and cinnamon) because they can be detected by anosmic individuals. The number of odorants may be important because the smell presentation kit or test may be repeatedly taken (e.g., each day for several weeks), it may include enough odorants so that people do not give rote answers. Uniformity in how the odorant is delivered may be important (e.g., odorant pens, scratch-and-sniff, etc.) and they may be easily accessed, without tools (e.g., coins which are often used for scratch-and-sniff) and without introducing new sources of variation (e.g., unequal scratching when releasing the odorant). Avoiding physical contamination may be important and participants in some instances may not share the same olfactory stimulus (e.g., odorant-containing pens to reduce the transfer of potential pathogens from nose to hand). Finally, the smell presentation kit or test structure may need to be robust against guessing. The systems, kits, and methods described herein, may have these benefits.

Some aspects include improving or changing a user's mental state. Some aspects include changing a user's appetite. Some aspects include providing the user a panel comprising: a plurality of patches. In some embodiments, each of the plurality of patches comprising odorous volatile compound having a prescribed distinct odor. In some embodiments, the subject or user performs an odor search among the plurality of patches. In some embodiments, the subject or user is instructed to perform the odor search. In some aspects, the test administrator will communicate instructions for an odor search. The instructions may be communicated to the user or subject. Some aspects include providing a user a panel comprising: a plurality of patches, each of the plurality of patches comprising odorous volatile compound having a prescribed distinct odor, and periodically quantitatively and/or qualitatively assessing the user's ability to correctly characterize the distinct odor, thereby assessing the user's mental state.

### Smell Assessment

Smell assessment may be used in conjunction with smell presentation. For example, a smell presentation program may be accompanied by periodic smell assessment, or by smell assessment before and after training. The smell assessment may be used to measure an increase in a subject's ability to smell, or may be used to identify or distinguish people with various smell disorders such as anosmia, hyposmia, or parosmia.

### Olfactory Test Kits

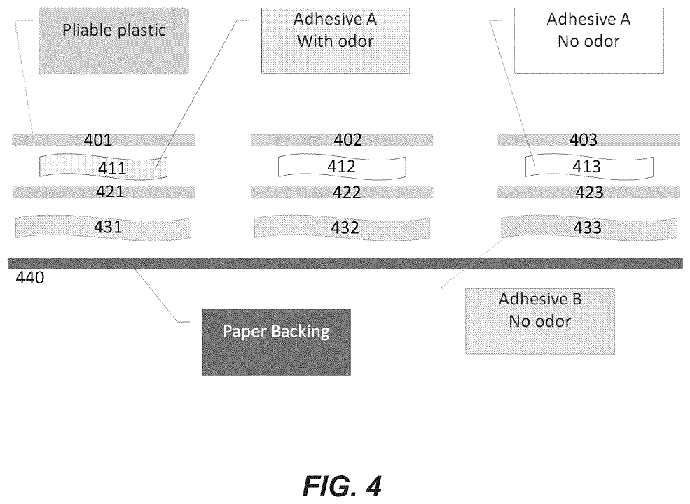

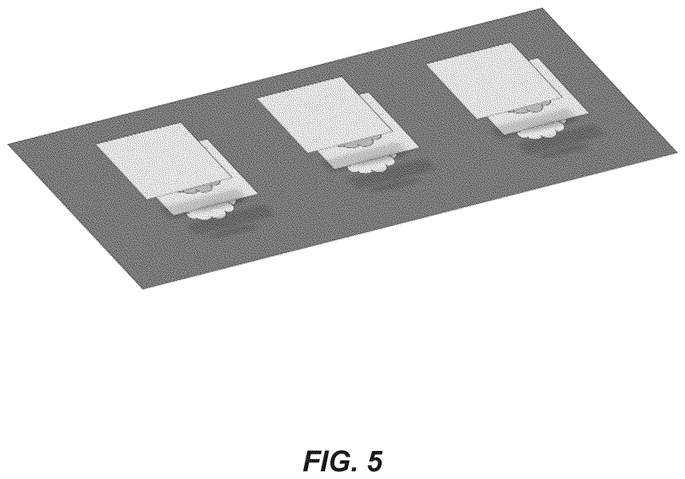

Disclosed herein, in some cases, are multifunctional smell assessment kits. The kit may include three odor labels adhered to a backing. The three odor labels may each include an outward surface (e.g., a flat plastic surface) adhered by an adhesive to another surface or backing. For example, the adhesive may be directly adhered to another surface (e.g., another flat plastic surface) that is directly adhered to a panel or backing (e.g., as shown or described in FIG. 4 or FIG. 5). Also possible are embodiments that include the adhesive directly adhered to the panel or backing. One of the odor labels may include an odorous compound within the adhesive, while the other two odor labels do not include the odorous compound. The odor labels may also be referred to as, or include, an odor sticker or smell sticker. The odor labels may include plastic, rubber, silicone, paper, or another material. The adhesive may include a glue, a resin, a rubber adhesive, an acrylic adhesive, or any other adhesive capable of retaining an odorous compound for use in the smell assessment. The panel or backing may include paper, card stock, plastic, rubber, metal, silicone, or another material. This kit may be used to retain the odor within the adhesive of one of the odor labels for an extended period of time, while releasing the odor to a user in a uniform, predictable way when the odor label is peeled away from the panel or backing.

The kit including the three odor labels bound either directly or indirectly to a backing allows for a simple system or method of smell assessment that includes three queries in any order to determine if a user has a smell impairment. The three queries may identify whether the user detects the odorous compound, whether the user identifies the odor of the odorous compound, and an intensity by which the user perceives the odor (if the user perceives the odor at all). The kit along with these three queries may aid in smell assessment without the need for a multitude of smells or other complexities.

Disclosed herein, in some cases, are odor kits for smell assessment. The odor kit may be a multifunctional smell assessment kit. The multifunctional smell assessment kit may comprise at least two surfaces (e.g., three surfaces) adhered to each other by a first releasable matrix (e.g., adhesive) which may contain an odorous volatile compound. The first surface adhered by a first releasable matrix comprising an odorous volatile compound may be referred to as an odor sticker or an odor label. The odor label may contain an odor released when the two surfaces are separated, or may be a blank, containing minimal odorous volatile compound. In some cases, only one of the surfaces is adhered using the adhesive with the odorous volatile compound, while the other(s) are adhered using the same or similar adhesive but without the odorous volatile compound. Some embodiments include a first and second releasable matrix (in any order). In some cases, the second releasable matrix may not release a volatile compound detectable through olfaction. The multifunctional smell assessment kit may comprise 1, 2, 3, 4, 5, 6, 7, 8, 9, 10, or more smell stickers. The multifunctional smell assessment kit may comprise 1, 2, 3, 4, 5, 6, 7, 8, 9, 10, or more odorous volatile compounds.

The first releasable matrix may be a first releasable matrix (e.g., adhesive). The second releasable matrix may be a second releasable adhesive. The first surface and/or the second surface may comprise plastic, rubber, silicone, or paper. The first or second releasable matrix may comprise a glue, a resin, a rubber adhesive, or an acrylic adhesive. The first releasable matrix may comprise an oil comprising the odorous volatile compound. The odor may comprise a familiar household odor. For example, the odor may comprise a rose, a popcorn, a coffee, orange, coconut, strawberry, banana, or a chocolate odor. Other examples include butter, fruit, lemon, soap, eucalyptus, gas, cloves, cinnamon, thyme, lavender, or honey.

The odor may be selected from a number of odors. For example, a group of smell tests may be manufactured that comprise any of 1, 2, 3, 4, 5, 6, 7, 8, 9, 10, 11, 12, 13, 14, 15, or more smells, wherein each smell test includes one smell (although some embodiments may include more than one smell per kit). This may allow the tests to be used without the subject being able to guess what the smell is, particularly if the subject has had the test before and know what one or more of the possible smells is. The system may include multiple queries that provide increased discrimination power that may allow for use of a limited number of smells within the group of smell tests. The group of smell tests may be manufactured that comprise no more than 1, 2, 3, 4, 5, 6, 7, 8, 9, 10, 11, 12, 13, 14, or 15, smells, wherein each smell test includes only one smell. For example, the group of smell tests may include no more than 4 or 8 smells, whereas existing smell tests may need a greater number of possible smells within a group of tests.

In some cases, the odor (e.g., odorous volatile compound) can comprise the odor of a healthy food item. A healthy food item can refer to foods that provide essential nutrients, such as vitamins, minerals, antioxidants, and dietary fiber, and promote good health. For example, fruits and vegetables (such as, but not limited to apples, oranges, berries, spinach, broccoli, carrots, and bell peppers) can be healthy food items. In some cases, the odor (e.g., odorous volatile compound) can comprise the odor of an indulging food item. Indulging food items can refer to foods that are considered treats or occasional indulgences due to their higher calorie, sugar, or fat content. Indulging food items may not offer significant nutritional benefits. For example, desserts, sweets, and processed snacks (such as, but not limited to cakes, cookies, pastries, ice cream, chocolate bars, chips, crackers, popcorn, and candies) can be indulging food items.

In some cases, the odor (e.g., odorous volatile compound) ranges in concentration. In some embodiments, the odor has a concentration of about from 0.1 mg/mL to 50 mg/mL, 0.1 mg/mL to 20 mg/mL, 0.1 mg/mL to 10 mg/mL, 0.5 mg/mL to 10 mg/mL, 0.5 mg/mL to 5 mg/mL, 0.5 mg/mL to 2 mg/mL, or 0.75 mg/mL to 1.25 mg/mL. In some embodiments, the odor has a concentration of about at least 0.1 mg/mL, 0.25 mg/mL, 0.5 mg/mL, 0.75 mg/mL, 1 mg/mL, 1.25 mg/mL, 1.5 mg/mL, 2 mg/mL, 2.5 mg/mL, 3 mg/mL, 4 mg/mL, or 5 mg/mL. In some embodiments, the odor has a concentration of about at most 0.5 mg/mL, 0.75 mg/mL, 1 mg/mL, 1.25 mg/mL, 1.5 mg/mL, 2 mg/mL, 2.5 mg/mL, 3 mg/mL, 4 mg/mL, 5 mg/mL, 10 mg/mL, 20 mg/mL, or 50 mg/mL.

The multifunctional smell assessment kit may further comprise a series of odor labels adhered to a panel to provide a support structure. The panel may comprise paper, card stock, plastic, rubber, metal, or silicone. The odor label may adhere the support structure while retaining the odorous volatile compound. In some cases, an intensity of the odor of the volatile compound does not diminish while the odor label adheres the first surface to the support structure for a period of time. In some cases, the intensity of the odor of the volatile compound does not significantly diminish when the first releasable matrix re-adheres the first surface to the panel after the first surface is being peeled away from the support structure and re-adhered to the support structure. The panel may comprise at least 1, 2, 3, 4, 5, 6, 7, 8, 9, or 10 surface areas configured to adhere a surface and/or a releasable matrix. The odor kit may comprise a peeling layer disposed on top of a surface area of the panel. In some cases, at least a portion of the peeling layer is removably coupled to the panel (or support structure). Uncoupling a portion of a peeling layer from the panel (or support structure) may release a volatile compound detectable through olfaction. The peel and smell technology described herein is useful because it keeps the odorous compound fresh. This way the compound or odor of the compound does not diminish quickly overtime, and the smell test can be used multiple times, or the odor may be delivered consistently. In some cases, the smell test may be re-used 1, 2, 3, 4, 5, 6, 7, 8, 9, 10, 15, 20, or 25 times, or more, or a range of times defined by any two of the aforementioned integers. The consistency of odor delivery allows for the use of an intensity query by which the user rates the intensity of smell detected or perceived. The panel or support structure may be a backing such as a paper backing.

In some embodiments, an odor label comprises or consists of a single surface and the releasable adhesive. The odor label may be adhered to a support panel by means of the adhesive. The releasable adhesive may include the odorous volatile compound. In some embodiments, an odor label comprises a smell sticker. In some cases, a smell sticker may be anhydrous or dry. In some cases, a smell sticker may be hydrated. In some cases, a smell sticker may comprise a binding agent such as an adhesive. In some cases, a smell sticker may comprise an organic solvent. In some cases, a smell sticker may comprise a stabilizing agent, antioxidant, or preservative composition. In some cases, a smell sticker may be a non-scratch smell sticker. In some cases, a smell sticker may be a scratch-free smell sticker. In some cases, a smell sticker may be a peelable smell sticker. In some cases, a smell sticker may be a revealed by lifting of a panel or sealing material. In some cases, a smell sticker may be a contact-free smell sticker. In some cases, a smell sticker may serve as a baseline for olfactory ability. In some cases, a smell sticker may be a medical standard of olfactory ability. In some cases, a smell sticker may be configured to test the threshold of olfactory ability of a subject.

In some cases, the kit includes three odor labels. The odor labels may be indirectly adhered to the panel. For example, three odor labels may be adhered to one or more other surfaces (e.g., plastic surfaces) adhered to a panel or backing. Some such embodiments include a first surface (e.g., odor label) adhered by an adhesive with an odorous compound to another surface that is directly adhered to the panel or backing. Some embodiments include a second and third surface (e.g., second and third odor labels, in any order) each adhered by an adhesive without the odorous compound to other surfaces that are directly adhered to the panel or backing.

Some embodiments include any of the components shown in FIG. 4. The kit may include a first surface 401 adhered by an adhesive 411 comprising an odorous compound (e.g., odor label 1). The kit may include a second surface 402 adhered by an adhesive 412 not comprising the odorous compound (e.g., odor label 2). The kit may include a third surface 403 adhered by an adhesive 413 not comprising the odorous compound (e.g., odor label 3). Odor labels 1-3 may all include the same adhesive but for the odorous compound. The adhesive 411 of odor label 1 may be further adhered to a fourth surface 421. The adhesive 412 of odor label 2 may be further adhered to a fifth surface 422. The adhesive 413 of odor label 3 may be further adhered to a sixth surface 423. The fourth surface 421 may be adhered by an adhesive 431 to a panel 440. The fifth surface 422 may be adhered by an adhesive 432 to the panel 440. The sixth surface 423 may be adhered by an adhesive 433 to the panel 440. The adhesives adhering the fourth, fifth, or sixth surfaces to the panel may all be the same type of adhesive as each other. The adhesives adhering the fourth, fifth, and/or sixth surfaces to the panel may all be the same type of adhesive as the adhesives of the first, second, and/or third odor labels but for the odorous compound. For example, in some cases only the adhesive of the first odor label contains the odorous compound. In some alternatives, the first, second, and third odor labels are all directly adhered to a single surface that is adhered to the panel, whereas in other cases the first, second, and third odor labels are all directly adhered to separate surfaces that are adhered to the panel. Note that the terms first, second, third, fourth, fifth, and sixth are used for convention, and do not necessarily denote a specific order. For example, the first, second, and third odor labels may be in any order, or in any placement on the panel.

In some cases, only one odor is used per smell test kit. For example, a backing may include only one odor label comprising an odorous compound, whereas previous smell tests may include more than one odor. A combination of multiple queries enables the use of only one odor.

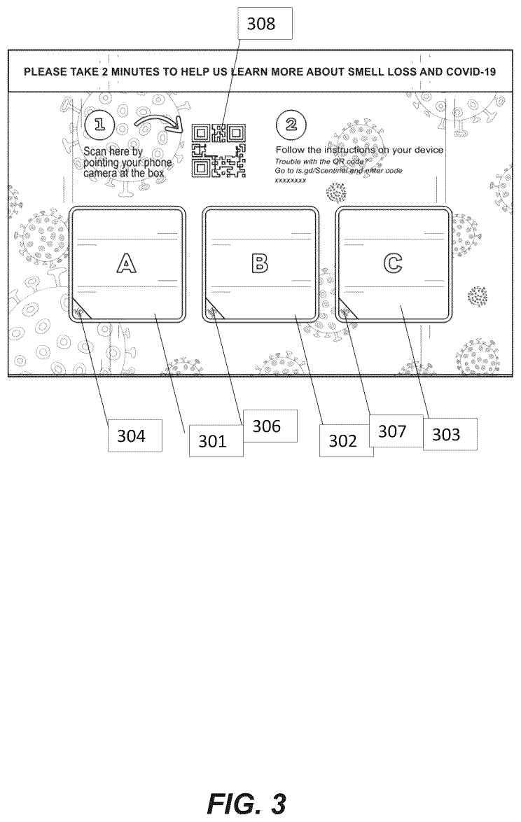

An example of an olfactory assessment kit is shown in FIG. 3, which shows a panel with a first surface adhered by a first releasable matrix 301, a second surface adhered by a second releasable matrix 302, a third surface adhered by a third releasable matrix 303. The first releasable matrix and the second releasable matrix may each comprise an odorous volatile compound. The third releasable matrix may not an odorous volatile compound. The third releasable matrix may a compound that is not odorous.

FIG. 3 also shows optional aspects such as peeling features and a symbol or code. The first releasable matrix 301 may be peeled using a peeling element 304. The second releasable matrix 302 may be peeled using a peeling element 306. The third releasable matrix 303 may be peeled using a peeling element 307. The panel may comprise a symbol or code 308.

The odor kit may further comprise a multifunctional smell assessment system. The system may comprise registering if the odorous volatile compound is detected by a user. The registering may comprise a first query for obtaining a first input on whether the user detects, by olfaction, the odorous volatile compound. The first input on whether the user detects, by olfaction, the odorous volatile compound may comprise a determination of which of the odor labels on the panel is considered odorous by the user. A user can consider any of the odor labels on the panel to attempt to detect, by olfaction, the odorous volatile compound of that surface. The user may consider a first surface or a second surface each comprising an odor of a volatile compound. The registering may comprise a second query for obtaining a second input on whether the user identifies the odor of the volatile compound. The second query may comprise a first image corresponding to the odor, and a second image not corresponding to the odor. The second input on whether the user identifies the odor may comprise a selection of the first image corresponding to the odor, or a selection of the second image not corresponding to the odor. The registering may comprise a third query for obtaining a third input on an intensity of the odor as perceived by the user. The third query may comprise a questionnaire associated with an intensity of the odor. The third input on the intensity of the odor as perceived by the user, may comprise a quantitation of the intensity of the odor. The queries need not occur in the order presented herein. For example, the second or third query may be presented to the user first, or the first or second query may be presented to the user last.

The multifunctional smell assessment kit may further comprise a network over which the first input is transmitted. The multifunctional smell assessment kit may further comprise a network over which the second input is transmitted. The multifunctional smell assessment kit may further comprise a network over which a third input is transmitted. The inputs may be obtained or transmitted in any order.

A user with a compromised sense of smell may be unable to detect or localize, by olfaction, the odorous volatile compound. The user with a compromised sense of smell may be unable to is unable to identify the odor. For example, a user with a compromised sense of smell may be unable to pick an image (e.g., image of a banana) associated with a smell (e.g., smell of banana). The user with a compromised sense of smell may be unable to perceives the odor below a designated intensity. The designated intensity may be at about 20 on a scale of 0-100, or may be at about 2 on a scale of 0-10. The designated intensity may be at least about 20 on a scale of 0-100, or may be at least about 2 on a scale of 0-10.

The query may be delivered orally, in written form, or by a computational device or telephone (e.g., smart phone). The system for registering if the odorous volatile compound is detected by a user may comprise a symbol or code. The symbol or code is printed on the panel. The symbol or code may comprise a quick response (QR) code or a web address. The symbol or code may be indicative of the composition of the odor labels. The symbol or code may be indicative that a first releasable matrix releases a first smell. The symbol or code may indicate a smell that a surface of a panel releases. The system may comprise a computer or an operator that may be configured to read the symbol or code to generate an output of a query.

The system may comprise a first output comprising the first query. The system may comprise a second output comprising the second query. The system may comprise a third output comprising the third query. The output may be a computer generate output.

The multifunctional smell assessment kit may further comprise a network over which the first output is transmitted. The multifunctional smell assessment kit may further comprise a network over which the second output is transmitted. The multifunctional smell assessment kit may further comprise a network over which a third output is transmitted.

The multifunctional smell assessment kit may further comprise a computational device comprising an output device configured to indicate an output. The output may indicate that the user may not have a compromised sense of smell when the first input indicates that the odorous volatile compound is detected by the user. The output may indicate that the user may not have a compromised sense of smell when the second input indicates that the odor is identified by the user. The output may indicate that the user may not have a compromised sense of smell when the third input indicates that the intensity of the odor is perceived by the user at or above the threshold intensity. The output may indicate that the user may not have a compromised sense of smell when the first input indicates that the odorous volatile compound is detected by the user, the second input indicates that the odor is identified by the user, and the third input indicates that the intensity of the odor is perceived by the user at or above the threshold intensity. The computational device may comprise a telephone (e.g., smart phone) or tablet.

The multifunctional smell assessment kit may further comprise a computational device comprising an output device, as described above, which is also configured to present the data (e.g., output) to a peer group. A peer group can refer to a collection of individuals within the same field or specialty who share similar levels of expertise, knowledge, and/or professional experiences. In some cases, the peer group can comprise a medical profession. In some cases, a peer group can comprise a group of medical professionals. For example, the peer group can be a group of medical professionals who can seek advice, share experiences, and discuss clinical challenges relating to the smell presentation system. In some cases, a peer group of medical professionals can play a role in improving patient (e.g., a subject and/or user) care and outcomes.

In some embodiments, a smell assessment kit comprises a questionnaire that determines the optimal odors to personalize a kit to a specific user. In some embodiments, the questionnaire comprises questions regarding sensitivities such as allergies or genetic predispositions to a volatile odorous compound. In some cases, the questionnaire is administered prior to beginning a training regimen. In some cases, the questionnaire comprises baseline or threshold assessment of a specific user's olfactory ability.

### Olfactory Testing Systems

Also disclosed herein, in some cases, are systems for multifunctional smell assessment. The system may comprise an odor kit. The odor kit may comprise a support structure comprising a first surface area. The odor kit may further comprise a first peeling layer disposed on top of the first surface area. At least a portion of the first peeling layer may be removably coupled to the support structure. The odor kit may further comprise a chemical substance disposed between the first peeling layer and first surface area. Uncoupling the portion of the first peeling layer from the support structure may release a volatile compound. The volatile compound may be detectable through olfaction. The system may further comprise a first module for receiving a first input from a user. The first input may correlate whether the user detected the volatile compound through olfaction upon uncoupling the portion of the first peeling layer. The first module may receive the first input through a user interface. The system may further comprise a second module for receiving a second input from a user. The second input may correlate whether the user identified a type of odor corresponding to the volatile compound upon uncoupling the portion of the first peeling layer. The second module may receive the second input through the user interface. The system may further comprise a third module for receiving a third input from a user. The third input may correlate to an intensity level provided by the user for the type of odor. The third module may receive the third input through the user interface. The system may further comprise a fourth module for determining an olfaction capability by the user based on the first input, the second input, and the third input. The fourth module can generate an output based on the determined olfaction capability. The first module, the second module, the third module, or the fourth module can be performed by a user.

The system may comprise a computer-implemented system. The computer-implemented system may comprise a first module for receiving a first input from a user. The first input may correlate whether the user detected the volatile compound through olfaction upon uncoupling the portion of the first peeling layer. The first module may receive the first input through a user interface. The computer-implemented system may further comprise a second module for receiving a second input from a user. The second input may correlate whether the user identified a type of odor corresponding to the volatile compound upon uncoupling the portion of the first peeling layer. The second module may receive the second input through the user interface. The computer-implemented system may further comprise a third module for receiving a third input from a user. The third input may correlate to an intensity level provided by the user for the type of odor. The third module may receive the third input through the user interface. The computer-implemented system may further comprise a fourth module for determining an olfaction capability by the user based on the first input, the second input, and the third input. The fourth module can generate an output based on the determined olfaction capability.

Also disclosed herein, in some cases, are smell detection systems. The system may comprise a panel comprising a first peelable surface adhered to the panel by a first adhesive matrix. The adhesive matrix may comprise an adhesive. The adhesive matrix may comprise a volatile compound configured to be detected by a person of ordinary olfactory sensitivity, and configured to be undetectable by a person having an impaired olfactory sensitivity. The first adhesive matrix may be an adhesive comprising the volatile compound. The compound may comprise an odor identifiable by the person of ordinary olfactory sensitivity and unidentifiable by the person having the impaired olfactory sensitivity. The system may further comprise a symbol or code identifiable by a computational device. The symbol or code may be configured to obtain a first input on whether the volatile compound is detected in the first adhesive matrix, a second input on whether the odor is identified, and a third input on a perceived intensity of the odor.

### Olfactory Test Methods

Also disclosed herein, in some cases, are smell assessment methods. The method may be used to identify an olfactory impairment in a subject. The subject may be suspected of having the olfactory impairment. The subject may have received a panel comprising a first peelable surface adhered to the panel by a first adhesive matrix. The first peelable surface and/or the first adhesive matrix may comprise an odorous volatile compound detectible by a person of ordinary olfactory ability, and undetectable by a person having an olfactory impairment. The compound may comprise an odor detectable by the person of ordinary olfactory ability and unidentifiable by the person having the olfactory impairment. The panel may comprise a second peelable surface adhered to the panel by a second adhesive matrix. The second peelable surface and/or the second adhesive matrix may not comprise an odorous volatile compound.

The method may comprise obtaining information about the subject. The information obtained may relate to a first question or instruction, a second question or instruction, and a third question or instruction (in any order). Some embodiments include obtaining information from the subject on whether the subject detects, by olfaction, the odorous volatile compound in the first adhesive matrix. Some embodiments include obtaining information from the subject on whether the subject identifies the odor of the volatile compound. Some embodiments include obtaining information from the subject on an intensity of the odor as perceived by the subject.

The method may comprise administering a questionnaire to a subject. The questionnaire may comprise a first question or instruction, a second question or instruction, and a third question or instruction (in any order). The first question or instruction may be designed for determining whether the subject detects, by olfaction, the odorous volatile compound in the first adhesive matrix. The second question or instruction may be designed for determining whether the subject identifies the odor of the volatile compound. The third question or instruction may be designed for determining an intensity of the odor as perceived by the subject. The first question or instruction may comprise a question or instruction to determine which of the first peelable surface and the second peelable surface the subject considers to comprise a stronger odor. The second question or instruction may comprise a question or instruction to determine which of a first image or a second image may comprise an image corresponding to the odor. In some cases, the first image may comprise an image corresponding to the odor. In some cases, the second image may comprise an image not corresponding to the odor. The third question or instruction may comprise a question or instruction to determine a quantitation of the intensity of the odor as perceived by the subject. The question or instruction to determine a quantitation of the intensity of the odor as perceived by the subject may comprise an image of a slider. The quantitation of the intensity of the odor as perceived by the subject may comprise a scale of 0-10. The quantitation of the intensity of the odor as perceived by the subject may comprise a scale of 1-10. The quantitation of the intensity of the odor as perceived by the subject may comprise a scale of 0-100. The quantitation of the intensity of the odor as perceived by the subject may comprise a scale of 1-100. In some cases, smell assessment methods comprise testing a subject to identify odors of a similar nature, such as in a class of odors such as citrus fruit (e.g., distinguishing lime and lemon odor). In some cases, the smell assessment methods comprise identifying a specific odor unique to a specific class of items. In some cases, a subject may be challenged to sample an odor without directly focusing their attention on such an odor. In some embodiments, a subject may be challenged to identify a potency of an odor. In some embodiments, a subject may be challenged to identify a threshold of detection of an odor. In some embodiments, a subject may be tested with identifying the pleasantness of an odor. In some cases, the smell assessment methods are repeated periodically such as for a week, two weeks, three weeks, or a month. In some cases, the smell assessment methods are repeated daily. In some cases, the smell assessment methods are repeated bi-daily, three times a day, or four times a day. In some cases, the smell assessment method comprises assessing (1) odor detection, (2) odor identification, (3) odor intensity, or any combination thereof. In some cases, the smell assessment method further comprises a therapeutic aspect comprising providing a therapeutic volatile odorous compound or compounds to a subject. In some embodiments, the therapeutic volatile odorous compounds reduce or increase appetite in a subject. In some embodiments, the therapeutic volatile odorous compounds reduce or increase body weight in a subject. In some embodiments, the therapeutic volatile odorous compounds reduce symptoms of a negative mental state such as PTSD or anxiety. In some embodiments, the smell assessment method further comprises operations to reduce symptoms of a negative mental state such as PTSD or anxiety. In some cases, the smell assessment method further comprises operations such as questionnaires regarding user mental states. In some cases, the smell assessment method further comprises operations such as gauging response time of a subject. In some cases, a specific volatile odorous compound provides therapeutic value. In some cases, a set or series of specific volatile odorous compounds provides therapeutic value.

The smell assessment method may further comprise determining whether the subject detects, by olfaction, the odorous volatile compound in the first adhesive matrix based on the subject's response to the first question or instruction, determining whether the subject identifies the odor based on the subject's response to the second question or instruction, and determining whether an intensity of the odor as perceived by the subject based on the subject's response to the third question or instruction.

The smell assessment method may further comprise identifying the subject as having olfactory impairment when the subject is determined to not detect, by olfaction, the odorous volatile compound in the first adhesive matrix, when the subject is determined to not identify the odor, or when the subject is identified to perceive the intensity of the odor as below a threshold intensity. The threshold may be obtained by comparing responses of subjects with normal senses of smell and subjects with compromised senses of smell, and may be adjusted in order to distinguish the two based on desired characteristics such as an optimal number of false positive or false negative values.

The smell assessment method may further comprise identifying the subject as not having an olfactory impairment when the subject is determined to detect, by olfaction, the odorous volatile compound in the first adhesive matrix, when the subject is determined to identify the odor, or when the subject is identified to perceive the intensity of the odor as at or above a threshold intensity.

The smell assessment method may further comprise identifying the subject as having olfactory impairment when the subject is determined to not detect, by olfaction, the odorous volatile compound in the first adhesive matrix, when the subject is determined to not identify the odor, and when the subject is identified to perceive the intensity of the odor as below a threshold intensity.

The smell assessment method may further comprise identifying the subject as likely to have a disease or disorder when the subject is identified as having the olfactory impairment, and identifying the subject as unlikely to have the disease or disorder when the subject is identified as not having the olfactory impairment. The disease or disorder may comprise an infection, a neurodegenerative disorder, or a cognitive impairment. The smell assessment method may further comprise administering to the subject a treatment such as a treatment described herein.

The smell assessment method may further comprise replacing the first peelable surface back on the panel, and re-using the first peelable surface adhered to the panel by the first adhesive matrix comprising the odorous volatile compound in an additional smell test. The additional smell test may be performed on a different subject. The additional smell test may be of the same subject at a later time than the first smell test. The smell assessment method may further comprise replacing the first peelable surface back on the panel, and re-using the first peelable surface adhered to the panel by the first adhesive matrix comprising the odorous volatile compound in 1, 2, 3, 4, 5, 6, 7, 8, 9, 10, 11, 12, 13, 14, or 15, or more, additional smell tests, or in a range of additional smell tests defined by any two of the aforementioned integers. The intensity of the odorous volatile compound may remain substantially constant in additional smell tests.

The smell assessment method may further comprise assessing the user's mental state before and/or after input on the user's physical health. In some cases, the method comprises assessing the user's mental state before and/or after input on the user's mood. In some embodiments, the method comprises assessing the user's mental state before and/or after input of the user's mental health status. In some cases, the method comprises determining whether the user's mental state has improved compared to a placebo or compared to the user's mental state prior to input on the user's physical health. In some embodiments, the method further comprises, assessing the user's mental state before and/or after exposing the user to the odorous volatile compounds. In some cases, the method further comprises, assessing the user's mental state before and/or after completing a smell presentation test. In some embodiments, the method further comprises, assessing the user's mental state before and/or after the user to completes the smell presentation test over a short exposure time, over a long exposure time, and/or at least twice daily over the course of at least eight weeks. In some cases, the method further comprises, determining whether the user's mental state has improved compared to a placebo or compared to the user's mental state prior to providing the user a panel.

Also disclosed herein, in some cases, are smell detection methods for detecting an olfactory sensitivity impairment. The method may comprise: (a) providing an odor kit to a subject, (b) uncoupling the portion of the first peeling layer from the support structure by the subject; and (c) administering a questionnaire to the subject. The odor kit may comprise a support structure having a first surface area; a first peeling layer disposed on top of the first surface area; and a chemical substance disposed between the first peeling layer and first surface area. At least a portion of the first peeling layer may be removably coupled to the support structure. Uncoupling the portion of the first peeling layer from the support structure may release a volatile compound detectable through olfaction. In some cases, the questionnaire may comprise a first question or instruction for determining whether the subject detects, by olfaction, the volatile compound upon the uncoupling of step b, a second question or instruction for determining whether the subject identifies a type of odor associated with the volatile compound, and a third question or instruction for determining an intensity of the type of odor as perceived by the subject.

The subject may be considered to fail the smell test if the subject fails 1 of the 3 queries (e.g., if the subject cannot (1) determine which smell label comprises the odor, (2) identify the odor, or (3) perceive the odor above a threshold intensity level). The subject may be considered to fail the smell test if the subject fails 2 of the 3 queries. The subject may be considered to fail the smell test if the subject fails 3 of the 3 queries. The subject may be considered to pass the smell test if the subject fails no more than 1 of the 3 queries. The subject may be considered to pass the smell test if the subject fails no more than 2 of the 3 queries. The subject may be considered to pass the smell test if the subject fails 0 of the 3 queries. In some cases, if the subject fails the test, the subject is considered to have an olfactory impairment. In some cases, if the subject does not fail the test, the subject is not considered to have an olfactory impairment.

Several methods disclosed herein may be facilitated with a multifunctional smell presentation tool. In one aspect, provided herein is a multifunctional smell presentation tool comprising one or more lift and smell odor panels comprising one or more odorous volatile compounds, wherein the one or more odorous volatile compounds may range in concentration, and a system for instructing users to interact with the one or more lift and smell odor panels based on one or more instructions in order to induce a user to receive one or more odorous volatile compounds amongst one or more distraction odors. In some embodiments, the multifunctional smell presentation tool is used with a test administrator communicating instructions for an odor-related task.

Provided herein, in some aspects, are methods for maintaining neurological function using the multifunctional smell presentation tool, wherein a test administrator further communicates instructions to prolong or repeat odor exposure. Provided herein, in so me aspects, are methods for enhancing neurological function as in cognition or memory using multifunctional smell presentation tool, wherein a test administrator further communicates instructions to prolong or repeat odor exposure. Provided herein, in some aspects, are methods for influencing eating behavior and diet quality or nutritional intake using multifunctional smell presentation tool, wherein a test administrator further communicates instructions to prolong or repeat odor exposure.

### Subjects and Treatment

A number of subjects may benefit from smell presentation, or from use of a smell test. Any subject with a smell disorder or impairment may benefit. Subjects with a healthy sense of smell may also benefit, for example by receiving confirmation of their healthy sense of smell or by further improving their sense of smell. The subject may be screened or monitored using the methods, systems, or kits described herein.

The subject may be an animal. The subject may be a vertebrate. The subject may be a mammal. The subject may be a primate. The subject may be a human. The subject may be male or female. The subject may be an adult.

The subject may be identified as having a compromised sense of smell or may be suspected of having a compromised sense of smell. The methods, kits, or systems described herein may be used to test or treat the compromised sense of smell. The compromised sense of smell may include anosmia, hyposmia, or parosmia. The subject may have anosmia. “Anosmia” may be used in the broad general sense, or in the narrow sense used medically. The subject may have hyposmia. The subject may have parosmia.

The subject may have a disease or disorder such as an infection, compromised psychological state, a neurodegenerative disorder, or a cognitive impairment. The subject may have an infection. The infection may result in a loss or lack of sense of smell in the subject. The infection may comprise a coronavirus infection. The coronavirus infection may comprise coronavirus disease 2019 (COVID-19). The coronavirus may include severe acute respiratory syndrome coronavirus 2 (SARS-CoV-2), or a variant thereof (e.g., an Alpha, Beta, Delta, or Gamma variant).

The subject may have a compromised psychological state. In some cases, the compromised psychological state is PTSD. In some cases, the compromised psychological state is anxiety. In some cases, the compromised psychological state is a state outlined in the Diagnostic and Statistical Manual-V of the American Psychiatric Association. The compromised psychological state may result in a loss or lack of sense of smell in the subject. The compromised psychological state may comprise anxiety, depression, stress, or post-traumatic stress disorder.

Anxiety can refer to an anxiety disorder. In some cases, anxiety disorders can refer to a class of psychiatric disorders that involve extreme fear or worry. Oftentimes, anxiety disorders can interfere with daily activities, such as job performance, schoolwork, and relationships. Anxiety disorders can have a variety of causes, including changes in the brain, environmental stress, and genetics. Non-limiting examples of anxiety disorders include acute stress disorder, anxiety due to a medical condition, generalized anxiety disorder, panic disorder, panic attack, a phobia, post-traumatic stress disorder, obsessive-compulsive disorder, separation anxiety disorder, social anxiety disorder, substance-induced anxiety disorder, or selective mutism.

The subject may have a neurodegenerative disorder. The neurodegenerative disorder may result in a loss or lack of sense of smell in the subject. The neurodegenerative disorder may comprise Parkinson's disease or Alzheimer's disease. The neurodegenerative disorder may include Parkinson's disease. The neurodegenerative disorder may include Alzheimer's disease.

The subject may have a cognitive impairment. In some situations, cognitive impairment is a result of experiences such as falls, car crashes, or accidental striking to the head. The cognitive impairment may result in a loss or lack of sense of smell in the subject. The cognitive impairment result from a head injury. Examples of head injuries include a collision head injury, such as may result from an automobile accident or sports injury. The head injury may comprise a traumatic brain injury or a head trauma.

The subject may have obesity. Obesity can be associated with health complications. For example, individuals with obesity can be at high risk of several physical diseases, such as certain cancers, diabetes, hypertension, heart disease, stroke, as well as being at increased risk of mortality. Obesity can be associated with mental health problems and psychosocial difficulties. For example, obesity can contribute to eating disorder behaviors and vice-versa. In some cases, the subject may have an eating disorder. The subject may have obesity and an eating disorder. Eating disorders can refer to irregular eating habits and/or severe distress or concern about body weight or shape. Eating disorders can refer to any of a range of psychological disorders characterized by abnormal or disturbed eating habits. For example, eating disorders may include inadequate or excessive food intake which can ultimately damage an individual's physical and/or psychological health. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof.

The methods described herein may include the user performing an odor search among the plurality of patches (from the smell presentation programs including devices, systems, kits, and/or methods provided herein) to momentarily change a mental state in order to provide a cognitive and/or behavioral therapy. For example, odor can be used as a sensory tool in cognitive or behavioral therapy to temporarily alter a person's (e.g., a subject and/or user) mental state or distract them from distressing thoughts or emotions. In some case, an odor being used as a sensory tool in cognitive or behavioral therapy to temporarily alter a person's mental state or distract them from distressing thoughts or emotions can refer to “scent distraction” and/or “olfactory distraction”. In some cases, the odor search among the plurality of patches can momentarily change a mental state by stimulating different senses, acting as a grounding technique (e.g., providing a sensory anchor to the present moment), creating positive associations (e.g., evoking powerful emotional responses and trigger memories), distracting from negative cognitive patterns, and/or enhancing relaxation and mindfulness (e.g., using aromatic scents, such as lavender or chamomile, for relaxation-inducing properties).

The methods described herein may include administering or recommending a treatment to a subject. The treatment may include the use of a smell presentation regimen. For example, the treatment may include the use of a smell presentation method described herein. The treatment may be related to the underlying cause of the subject's smell defect. The treatment may include a coronavirus treatment. The method may include a neurodegenerative disease treatment. The method may include a head injury treatment. The method may include a cognitive impairment treatment. The method may include a weight gain treatment. The method may include a weight loss treatment.

The treatment may comprise a pharmaceutical composition. The pharmaceutical composition may be administered orally, by injection, or by another route. The treatment may include a surgery. Examples of surgeries include a head surgery, a nose surgery, or a brain surgery. The treatment may include monitoring or watchful waiting.

In some cases, the treatment may include inclusion or exclusion of the subject from some activity. For example, the treatment may include exclusion from a scenario or situation of risk to the subject or risk of transferring an infection from the subject to uninfected subjects. In some cases, a person may be allowed or denied entry into a building or public space based on an aspect described herein.

### Computer Systems (e.g., Electronic Systems)

The present disclosure provides computer systems (e.g., electronic systems) that are programmed to implement methods or systems of the disclosure (such as a method for smell presentation or smell testing, or a system for smell presentation or smell testing). The present disclosure provides electronic systems that are programmed to implement methods or systems of the disclosure (such as a method for smell presentation or smell testing, or a system for smell presentation or smell testing). For example, a system (e.g., electronic systems) may include a communication interface that receives data over a communication network. The data may include input, such as input from a user, or input from a medical practitioner. The system (e.g., electronic systems) may further comprise a computer in communication with the communication interface, wherein the computer comprises one or more computer processors and computer readable medium comprising machine-executable code that, upon execution by the one or more computer processors, implements a method described herein. The electronic systems may further comprise a computer in communication with the communication interface, wherein the computer comprises one or more computer processors and computer readable medium comprising machine-executable code that, upon execution by the one or more computer processors, implements a method described herein. For example, the computer may implement a method that includes receiving data such as inputs, or outputting information to a user or to a medical practitioner. The computer may be used in conjunction with a smell test or a smell presentation program described herein.

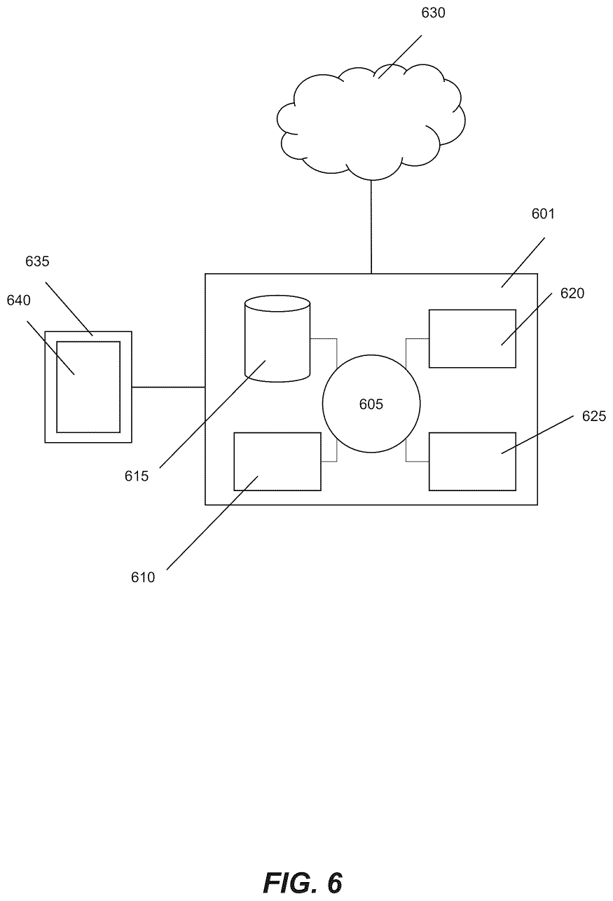

FIG. 6 shows a computer system 601 that is programmed or otherwise configured to perform methods described herein. The computer system 601 can regulate various aspects of the present disclosure, such as, for example, receiving a symbol or a code to identify a panel, provide a first, a second, or a third query to obtain a first input, a second input, and/or a third input, generate a first, a second, or a third output, and provide an assessment, recommendation, or other output related to smell testing or training. The computer system 601 can be an electronic device of a user or a computer system that is remotely located with respect to the electronic device. The electronic device can be a mobile electronic device. In some cases, a first input device may be a touch screen, keyboard, or mouse. In some cases, a second input device may be a camera for the code or symbol. The touch screen or other device may include a GUI that has a slider tool (e.g., a ruler type bar where a person moves his or her finger across the screen to select the intensity of smell detected). First, second, and third may be used for convention, and the queries, inputs, and outputs may be in any order.

The computer system 601 includes a central processing unit (CPU, also “processor” and “computer processor” herein)605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), specifically designed kiosks, or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM. DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a symbol or a code to identify a panel, provide a first, a second, or a third query to obtain a first input, a second input, and/or a third input, provide a first, a second, or a third output, and provide an assessment or recommendation. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, receive a symbol or a code to identify a panel, provide a first, a second, or a third query to obtain a first input, a second input, and/or a third input, generate a first, a second, or a third output, and provide an assessment or recommendation. In some cases, an algorithm is used to adjust odor testing based on subject performance. In some cases, an algorithm is used to adjust difficulty of odor identification. In some embodiments, an algorithm is used to prioritize testing of odors. In some embodiments, an algorithm is used to prioritize testing of particular odors. In some embodiments, an algorithm is used to help a subject evaluate an odor. In some embodiments, an algorithm is used to adjust intensity of odor presentation. In some embodiments, an algorithm is used to adjust intensity of odor presentation dynamics. In some embodiments, an algorithm is used to develop an appropriate threshold test for a subject. In some embodiments, an algorithm is used to develop an appropriate concentration of an odorant molecule. In some embodiments, an algorithm is used to produce a potent concentration of an odorant molecule for a given subject. In some embodiments, an algorithm is used to produce a subtle concentration of an odorant molecule for a given subject.

Methods and systems of the present disclosure can compare data collected and/or inputted to a reference. For example, the electronic system provided herein can compare data collected and/or inputted to a reference. In some cases, the reference can be a control. The control and/or reference can be obtained from a database. For example, the control and/or reference can be acquired from public health databases. The databases can include databases maintained by public health agencies, research institutions, and other organizations with a focus on public health. For example, the control and/or reference can be obtained from Centers for Disease Control and Prevention, World Health Organization, National Institutes of Health, European Centre for Disease Prevention and Control, and/or Public Health England. The information obtained from the database can be health-related information. The information obtained from the database can be collected, stored, and/or disseminated in order to acquire a suitable reference and/or control. Additionally, the control and/or reference can be the user's mental state before and/or after input on the user's physical health. In some cases, the control and/or reference can be user's mental state before and/or after input on the user's mood. In some embodiments, the control and/or reference can be the user's mental state before and/or after input of the user's mental health status. In some cases, the control and/or reference can be from a placebo control. In some cases, the control and/or reference can be the user's mental state prior to input on the user's physical health. In some embodiments, the control and/or reference can be the user's mental state before and/or after exposing the user to the odorous volatile compounds. In some cases, the control and/or reference can be the user's mental state before and/or after completing a smell presentation test. In some embodiments, the control and/or reference can be the user's mental state before and/or after the user to completes the smell presentation test over a short exposure time, over a long exposure time, and/or at least twice daily over the course of at least eight weeks. In some cases, the control and/or reference can be the user's mental state prior to providing the user a panel.

### Web Application

In some cases, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some cases, a web application is written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or eXtensible Markup Language (XML). In some cases, a web application is written to some extent in a presentation definition language such as Cascading Style Sheets (CSS). In some cases, a web application is written to some extent in a client-side scripting language such as Asynchronous Javascript and XML (AJAX), Flash® Actionscript, Javascript. or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some cases, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB.NET, or combinations thereof. Compilation may be often performed, at least in part, to create an executable program. In some instances, a computer program includes one or more executable complied applications.

### Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

In some cases, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Module

The medium, method, and system disclosed herein frequently comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some cases, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Database

The medium, method, and system disclosed herein can comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some cases, a database is internet-based. In some cases, a database is web-based. In some cases, a database is cloud computing-based. A database may be based on one or more local computer storage devices.

### Data Transmission

The subject matter described herein, including methods for smell presentation or assessment, are configured to be performed in one or more facilities or at one or more locations. Facility locations are not limited by country and include any country or territory. In some instances, one or more steps are performed in a different country than another step of the method. In some cases, one or more method steps involving a computer system are performed in a different country than another step of the methods provided herein. In some cases, data processing and analyses are performed in a different country or location than one or more steps of the methods described herein. In some cases, one or more articles, products, or data are transferred from one or more of the facilities to one or more different facilities for analysis or further analysis. An article may include one or more panels used for testing or training. In some embodiments of the methods and systems described herein, the analysis is performed and a subsequent data transmission step will convey or transmit the results of the analysis.

In some cases, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, performed in one facility in a particular location and corresponding data transmitted to another location, or directly to an individual, such as data related to a smell presentation regimen, a smell test, a diagnosis, a prognosis, or the like, in the same or different location or country.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating.” “assessing.” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “user” are often used interchangeably herein. A “subject” can be an animal with a nose or sense of smell. The subject can be a mammal. The subject can be a primate. The mammal can be a human. The subject may be a patient. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “test administrator” refers to an individual aiding in the administration of a tool or system disclosed herein, performing tasks such as adjusting intensity, odor type, switching panels, or changing odor detection threshold parameters.

As used herein, the term “identify odor” refers to an individual's ability to connect an odor with a naturally correlated object or phenomenon.

As used herein, the term “source” refers to a naturally correlated source of an odor, such as orange being a source of an odor of orange.

As used herein, the term “reference” refers to an established identify of an odor, such as an internal standard of a particular smell.

As used herein, the term “gamified” refers to a stratification of an application or process wherein a subject or user is rewarded directly or indirectly as the subject or user progresses within the application or process.

As used herein, the term “smell presentation system” is a device, system, or tool for providing volatile odorous compounds or mixtures to a user. A smell presentation system may provide channels for input of user information, as well as outputs adjust for the purpose of a given user.

As used herein, the term “assessing” refers to a process of evaluating a set of data or information such as a user's response to a set of volatile odorous compounds.

As used herein, the term “mental state” refers to a set of emotional, physiological, and/or spiritual status of an individual or subject. Examples of a negative mental state include anxiety, fear, trauma-induced emotions, paranoia, and so forth. Further examples of mental states are outlined in the American Psychiatric Association's Diagnostic and Statistical Manual-V.

As used herein, the term “specific odor” refers to a single volatile organic compound or set of volatile organic compounds naturally correlated with each other.

As used herein, the term “therapeutic” refers to a property of reducing a negative attribute in a subject, including medical attributes.

The section headings used herein are for organizational purposes only and are not to be construed as limiting the subject matter described.

### EXAMPLES

The following examples are included for illustrative purposes only and are not intended to limit the scope of the invention.

### Example 1: a Rapid Test to Screen for Smell Loss or Assess Smell Presentation

Smell tests can be costly and can be lengthy for population surveillance in health emergencies. To fill this gap, a smell test which evaluates smell in three ways in less than two minutes was developed. Its performance in discriminating between people with little or no sense of smell (anosmics) and individuals with other smell disorders from those with an average sense of smell (normosmics) was assessed. Also assessed was whether the smell test would perform similarly to the NIH Toolbox® Odor Identification Test in normosmics.

The smell test, described herein, measures olfaction by detection, intensity, and identification and the responses are collected via an online portal. Using Bayesian linear models, we compared the three outcome measures in adults with anosmia (N=111) and people with other smell disorders (e.g., hyposmia, parosmia, N=42) to people with no known problems in their sense of smell (N=154).

### Participants

Eligible participants were recruited via a flyer distributed through the Monell Newsletter, allowing the enrollment of normosmic subscribers and subscribers with smell loss. Volunteers completed an eligibility survey in which they reported their age (inclusion criteria: 18-75 years old, 265 excluded), whether they had access to a smart device (phone or tablet) or a computer (6 excluded), and whether they were currently residing in the United States (121 excluded).

A total of 532 smell tests were distributed by mail on a first-come, first-served basis; 308 participants reporting no history of smell problems received one smell test, and one NIH Toolbox® Odor Identification Test. Participants with pre-existing forms of smell loss (N=224) received one smell test, and were not asked to complete the NIH Toolbox® Odor Identification Test to limit the frustration generated by being confronted with smell tasks. Participants were also invited to take the smell test (and the NIH Toolbox® Odor Identification Test, if available) on the same day they were scheduled to have a COVID-19 PCR test. Participants were then asked to report the results of the COVID PCR test via survey when the outcome was known. Only 3 participants took the smell test/s and the COVID PCR test, thus given the low numerosity this data is not shown. The response rate from completion of the eligibility survey was 79%, with a final sample size of participants who consented and completed the study including 154 normosmic adults, 111 anosmics and 42 participants with other smell disorders [fluctuations (N=5); hyposmia (N=23), parosmia (N=5), other (N=4), COVID-related smell loss (N=3)]. Given the low numerosity in each category of the smell disorders variable, no separate statistical analyses were performed on this factor. The normosmic participants who completed the NIH Toolbox® Odor Identification Test were 148.

### Procedure

The study started on Sep. 4, 2020 and was completed by Sep. 15, 2020. During this time, participants were contacted via the Monell Newsletter, and completed an 10-question online eligibility survey. If they were not eligible or if they responded after the target number of participants had been enrolled, they were thanked and informed that they would not be enrolled in the study. If, on the contrary, they were deemed to be eligible, they received one or two smell tests via mail depending on their anosmic/normosmic self-report. Once participants received the test, they had to complete them within the next 14 days.

Participants used a QR-code or a web address to access the RedCap survey used to record self-reports on demographic data (age, gender, ethnicity), pre-existing smell and taste loss as well as complete the smell test and the NIH Toolbox® Odor Identification Test, if available.

To complete smell test, the instructions were to (a) consecutively open one odor patch at a time, smell it and reseal it; (b) choose the strongest odor-containing patch from three patches, two of which do not have an odor; (c) rate on a scale from 0 to 100 the intensity of the odor detected; and (d) select the best verbal and visual label for the odor among four verbal and visual options. If the participant gave an incorrect response to (d), they were instructed to try again to identify the odor, this time among the three remaining options.

No feedback was provided on the accuracy of the odor identification after the second attempt. Intensity was included because a cutoff rating (e.g., <20 on a 1-100 scale) can be predetermined to signal smell loss for an odorant generally perceived as moderate to strong, and useful for tracking an individual's smell function over time (i.e., identifying changes with repeated testing). Additionally, it can be highly discriminative for COVID-19 infection.

The group of participants (normosmics) who also completed the NIH Toolbox® Odor Identification Test were instructed to scratch and sniff each of the 9 odors included in the NIH Toolbox® Odor Identification Test and identify among 4 visual and verbal options which one corresponded to the odor smelled. Participants identified a total of nine odors. Subsequently, the participants completing the NIH Toolbox® Odor Identification Test could opt in to answer questions regarding their health status, with particular reference to COVID-19 and other respiratory illnesses. Although no formal data were collected on the completion time of the smell test, a limited number of participants (N=10) reported that the test takes ˜2 minutes to complete when including the demographic questions and <1 minute to complete the smell test smell subtests.

### Statistical Analysis

The cross sectional design included a between-subject factor “Smell ability” (anosmic, other smell disorders, and normosmics) and within-subject factors, namely the scores at the subtests of smell test (odor discrimination, odor intensity, odor identification) as well as the smell assessment score (anosmia/normosmial), and the scores at the single items and the total score at the NIH Toolbox® Odor Identification Test.

Each smell test subtest returned one of the following scores: odor detection accuracy (correct/incorrect); odor intensity, (above/below a cut off of 20) and odor identification among 4 given options (correct/incorrect), and when the first response was incorrect, among the three remaining options (correct/incorrect). The NIH Toolbox® Odor Identification Test returned two scores: the official scoring (anosmia=<=3; hyposmia=4-6; normosmia>=7 (Dalton et al., 2013)) and a binarized version of the official score to directly compare the smell test score (anosmia=<=4: normosmia>=5).

A Sequential Bayes Factor design (SBFD) with maximal N was used. This may maximize the probability of obtaining the desired level of evidence and a low probability of obtaining misleading evidence. Additionally, this SBFD design may require on average half the sample size compared to the optimal null hypothesis testing fixed-n design, with comparable error rates. A desired grade of relative evidence for the alternative vs. the null (BF10) hypothesis was set at BF10>6 (moderate evidence) for H1 and BF01>3 for H0 (anecdotal evidence). Based on a conservative Cohen's D=0.5, a minimum sample size per group of n0=43 was specified. Once n0 was reached, the BF was computed on the existing data. BF computation continued after every participant was added (in the smallest or slowest accumulating group at that time) until the thresholds of H1 or H0 were reached, at which point sampling ceased. The main driver of the stop rule was, however, a time limit (September 15th). To test the hypotheses and explore the effect of covariates (age, sex, ethnicity), Bayesian linear mixed models using the BayesFactor package were used in the R Environment for Statistical Computing (R Core, 2020). Given the unequal distribution of the data across categories in the ethnicity variable, the responses were binarized as White/Non-white. To assess the differences in accuracy among tests and subtests, Bayesian and parametric tests were employed for equality of proportions with or without continuity correction.

### Results

A small group of anosmics (N=11, 10%) successfully completed the smell test. On the contrary, the majority of individuals with other smell disorders (N=27, 64%), as well as the vast majority of normosmics successfully completed the smell test (N=145, 94%). Participants from the three groups differently used the response strategies to complete the smell test. In the anosmic group, 23% of participants did not provide the correct response to any of the subtests, 41% did not successfully complete two subtests, and only 11% did not report an odor intensity above 20/100. In the other smell disorders group, 17% participants did not report an odor intensity above 20/100, 17% failed two out of the three subtests and only 2% did not provide the correct response to any of the subtests.

The combined accuracy at all three subtests may significantly discriminate the performance across the three groups. In particular, presently, the odor intensity score demonstrated a perfect ability to classify normosmia. In some cases, the only subtest that may not significantly discriminate between the performance of the three groups is the second odor identification, which was only used by a total of 32 participants across the three groups.

An identification test was used in order to compare the performance of the smell test against a validated smell test. Results indicated that the performance at both tests was concordant when considering the odor identification of the flower odor, which was item #9 in the NIH Toolbox® Odor Identification Test (143/148, 97% participants correctly identified the flower odor) and the smell assessment odor identification subtest (136/148, 92% accuracy in the first identification attempt). A 2-sample test for equality of proportions with continuity correction confirmed a lack of statistical difference between the two test scores (X2=2.25, df=1, p=0.13). In 17/148 cases (12%) the NIH Toolbox® Odor Identification Test and the smell test were discordant; in 12 cases the participant passed the NIH Toolbox® Odor Identification Test but failed the smell test; in 5 cases the participant passed the smell test but failed the NIH Toolbox® Odor Identification Test.

When considering the full NIH Toolbox® Odor Identification Test (9 items) and smell test (detection, intensity and identification) the classification accuracy converged: 92% of normosmics passed both tests.

No effect of age (BF10=0.81±0.02%), sex (BF10=0.84±0.02%) or ethnicity (BF10=0.48±0.02%) was retrieved for the performance at the NIH Toolbox® Odor Identification Test.

Results from this study showed that smell tests described herein may be used as a quick, practical, and direct test to distinguish between people with and without smell loss. It may also distinguish smell disorders useful in many situations such as COVID-19 symptom screening. Clinical applications and population surveillance that smell assessment promotes are multiple, including the early detection of psychiatric, neurological and neurodegenerative disorders, such as schizophrenia, traumatic brain injury, Parkinson and Alzheimer's disease. This test may further be used to assess smell presentation in a subject, such as before and after the subject undergoes smell presentation. This test may also be used to identify subjects in need of smell presentation.

### Example 2: Description of a Smell Test that May be Used to Assess Smell Presentation or Identify a Need for Smell Presentation

Three components of olfactory function were combined to develop a rapid, accurate and inexpensive smell screening test. The test was developed with multiple odorants that have high familiarity in the US-population. Combining three objective measures made a fast, and accurate and inexpensive smell test.

There are validated measures to ascertain for smell ability (e.g., NIH Toolbox), but they are developed to determine the presence of smell disorders following quite involved procedures. To deploy a smell-based surveillance for COVID-19, a fast method would be useful, for instance, for screening for entry into the workplace or school, speed is important to prevent bottlenecks, and create groups of people. Currently, even the fastest available validated smell tests of the validated methods may be too time-consuming.

It may be important to include one or more components of olfactory function that can be objectively assessed so that we have a falsification metric and the ability to calculate the probability of passing the test in the absence of smell. The test includes 3 tasks:
A detection task, in which only one out of three samples contain an odor, (e.g., pick the strongest odor-containing patch from three patches, two of which do not have an odor), which has a guessing probability of 33%
Odor identification in which the individual needs to select the best label for the odor, (pick the most suitable picture/label for the odor out of a choice of 4)
An intensity rating on a 0 to 100 scale with a cut-off rating (i.e., <20 on a 0-100 scale) to signal smell loss for an odorant that is generally perceived as moderate to strong.

The smell test, referred to as “SCENTinel,” comes in 8 different iso-intense fragrances, which allows for longitudinal within-subject testing for at least two working weeks, while providing enough variability to control for odor expectation as well as the rare possibility of specific anosmias. The fragrances allow for flexible use of the test across developmental stages, from children of 3 years old to the elderly, and from other smell identification tests standardized in the US population

Given the fast nature of the test, a peel and smell sticker technology, which encapsulates the fragrance on a patch that delivers scent only when the top layer is lifted. This technology prevents cross-contamination across odor samples on the same card (useful for an accurate odor detection discrimination test) and allows the standardization of odor delivery across cards and odors (useful for an accurate odor intensity test), and limits the odor residual in the air post-test, which could greatly compromise the ability to identify the odors in an odor-saturated environment. Strategies like having a trained technician deliver the test (like in the NHANES case), represents an increased risk of contagion for person, and may not fully avoid that different test results may be confounded by different ways of scratching the odor patch. Relying on instructions provided to untrained participants may also not guarantee within and between participant administration standardization and as a consequence, the stability of the results may be affected. Pilot data collected with the peel and smell technology showed that the SCENTinel test takes most people less than two minutes to complete.

The components of the SCENTinel test are:
A test backing material on which the odor labels and instructions are applied. The backing is card stock material or copy paper.
3 odor labels, one with an odor, two blanks. The odors are scented oils. The labels are produced in rolls like address stickers which are then adhered to the backing.
A software program to calculate the results. The user scans a QR code or enters a web site to complete an online survey. The survey asks a series of questions and the results are either returned to the user or added to a database. The survey in its current form is written in RedCap. REDCap is a secure web application for building and managing online surveys and databases. While REDCap can be used to collect virtually any type of data in any environment (including compliance with 21 CFR Part 11, FISMA, HIPAA, and GDPR), it is specifically geared to support online and offline data capture for research studies and operations.

This test may be used to assess smell presentation in a subject. For example, the test may be administered to a subject after a subject undergoes smell presentation to assess if the subject's smell has improved by the training.

The test may further be used to identify a subject in need of smell presentation. For example, a subject may be identified as in need of training when the subject is identified as having an impaired sense of smell using the smell test. The subject may then be administered smell presentation regimen.

### Example 3: Use of SCENTinel Combined with Smell Presentation

A smart phone application (“app”) prompt a subject with a compromised sense of smell to undergo a smell presentation session. The app indicates which of several smell cards of a smell presentation kit to use in the session. The app also records information on the subject's overall health and mood. This allows for a portable and discrete smell presentation kit which can be performed at home or in a doctor's office.

First, a SCENTinel test as described in Example 2 is used to determine a baseline level of smell in the subject.

Olfactory training using a SCENTinel trainer is performed for 8 weeks. The subject sniffs each of four different odors on a module for at least 15 seconds each twice a day. The training occurs at least 30 minutes before or after a meal. The first module includes a smell card that includes a rose odor, a eucalyptus odor, a lemon odor, and a cloves odor, another smell card that includes a cinnamon odor, a thyme odor, a chocolate odor, and a peach odor, as well as other smell cards that include other odors. An additional smell card includes a coffee odor, a lavender odor, a honey odor, and a strawberry odor. Each training card may be used up to 14 times. The system prompts the user to let him or her know which training card to use at any given time during the training period.

After 8 weeks, a SCENTinel test is performed to assess progress. If the subject's sense of smell has reached an acceptable threshold, training can stop if desired. If not, the subject completes SCENTinel training with a second module for another 8 weeks. The second module includes smell cards that include additional odors, some stronger. The odors in the second module may be greater in intensity compared to the odors used in first module if needed for the subject to regain his or her sense of smell, or may be less in intensity compared to the odors used in first module if the subject is making progress but wants to continue to refine his or her sense of smell without the odors being as obvious.

After 8 weeks, a SCENTinel test is performed to assess further progress. If the subject's sense of smell has reached an acceptable threshold, training can stop. If not, the subject completes SCENTinel training with the third module for another 8 weeks. The third module includes additional odors that may or may not be included at differing intensities relative to the first or second modules.

After 8 weeks, a SCENTinel test is again performed to assess progress. If smell has reached an acceptable threshold, training can stop. If not, smell presentation resumes with the first module.

In addition, the subject is treated for an underlying condition (e.g., COVID-19 or a neurological disorder) that affects his or her sense of smell. Once the underlying condition is treated, the subject may regain some sense of lost smell that the smell presentation modules improve.

### Example 4: Superiority of Smell Presentation Stickers Compared to Other Methods

A subject (e.g., with COVID-19 or other disorders) suffers from smell loss. The subject desires to regain his or her sense of smell to full capacity and decides to start smelling things to try and identify odors. The subject begins to try smelling household kitchen items such as an orange, only to discover by being informed by someone who has not lost his or her sense of smell that the orange does not carry an odor because it is no longer fresh.

Because of these problems, the subject turns to the use of a smell presentation kit described herein. The smell presentation kit includes 4 smell stickers which, due to a protective layer over each adhesive matrix of the smell stickers, reliably retain their odors and are able to be used by the subject in a consistent manner.

Over time, the subject begins to regain his or her sense of smell, and begins using advanced smell presentation kits, each with labels indicating that they contain a lower intensity odor. The subject regains his or her sense of smell by gradually learning to smell, identify, and rate the intensity of progressively lower intensity odors. After regaining his or her sense of smell, the subject continues using smell presentation kits that have distinct wine smells, and goes on to become a leading wine expert, able to distinguish wine varieties by their various odors.

### Example 5: Traumatic Brain Injury Testing Kits

According to University of Pittsburgh Medical Center Sports Medicine Concussion Program, up to three million sports and recreation-related concussions (mild TBIs) are sustained each year and about half go unreported or undiagnosed. In a 2018 survey of military personnel, almost half of concussions were not reported. Not all patients with concussions appear to be acutely ill after a substantial physical blow. This can be a serious problem because after sustaining a concussion, the risk of sustaining another is as high as three-fold. Populations at risk for significant physical and/or social consequences from undiagnosed or late diagnosed concussions include athletes, military personnel and anyone involved in an accident. In order to prevent more severe and long-term sequelae, more timely screening for TBI is needed. The current methods for screening and diagnosing TBI are suboptimal because they are either not appropriate for on-site evaluation, not accessible/affordable for all patients, or slow. Novel and accessible tools that can perform an on-site, fast evaluation are needed for a timely diagnosis of TBI and avoidance of further sequelae.

Olfactory dysfunction can be a marker for consequences that present in the future, and smell dysfunction is a harbinger of TBI. The TBI testing device is a fast and easy-to-use screening device for evaluating cognition by assessing olfactory function [the first cranial nerve (CN-I)]. Fast identification of a possible TBI will reduce the risk of further injury and promptly directs patients to appropriate follow-up examination. This device is ideal for situations following at-risk contact when time to assess is limited, such as on a sporting field, at the scene of an accident, or in military conflict scenarios. The starting prototype device will include several “lift & smell” odor patches on a slender, portable, pocket-sized structure that is specifically designed for on-site use (e.g., light, flexible, resistant to liquids, no batteries required). In a minute or less, the possibly concussed patient smells the patches and a medical professional such as athletic trainer, nurse, emergency medical technician (EMT)] will administer questions that are printed on the device to assess olfactory functions such as detection, identification, odor memory and verbal/visual links to odors.

Odor patches are used because many patients do not recognize their olfactory dysfunction. The odor patches that are part of our new device will be standardized for intensity, culturally relevant, and vary so that guessing the odors will not be possible. Odors will be specifically selected not to generate false positives from trigeminal nerve stimulation. The device will be designed so no special training will be required to administer the test.

Olfactory dysfunction is usually measured in time since injury by days, weeks, or months, not within hours of a TBI. The Lecuyer Giguére study stands out because an olfactory assessment was conducted approximately 24 hours following the injury. Because olfactory neurons regenerate relatively quickly, valuable insight is lost when the first measurement of olfactory function is two weeks or more post-injury. The lack of fast and easy screening on-site may be thwarting the opportunity to gather data to better understand how olfactory function can predict the risk of future serious outcomes following a TBI. In a current embodiment, the device can measure olfactory function within hours following an injury.

Despite the serious consequences, there is a high percentage of unreported mild TBIs. That reality signals a clear need for change. A screening device such as the one provided herein will develop reduces barriers to assessing suspected concussions and provides a clear indicator for follow-up examination by a medical professional qualified to diagnose TBI. There are a variety of approaches for assessing TBI but none are ideal for on-site testing and many may not be accessible to all patients. Imaging and plasma tests are not appropriate for field use. Paper and app scored tests are time consuming and not practical for a fast, on-site evaluation. Portable devices that require batteries, LCD and special training to operate also present similar barriers. Furthermore, many of these alternatives are expensive, creating a financial barrier and healthcare disparities for local sports programs and EMS groups. TBI is diagnosed only by a qualified medical professional. Neuroradiological tests help doctors see brain injury but are rarely used for mild TBI. Plasma, EEG and infrared testing may have a place in the distant future but will require extensive modifications to be useful on the site of an accident.

Through this test, a low cost, easy to administer protocol that assesses a patient's olfactory function can be completed in one minute or less at the site of injury. With such a device, EMTs and other similar healthcare providers can assess the patients who may be in an at-risk accident although do not appear to have sustained a TBI. The anticipated outcome is the identification of more TBIs and reduction in unexpected long-term sequelae.

### Example 6: Algorithm Assisted Tests of Olfactory Ability

A subject undergoing tests of olfactory ability can be challenged at different levels depending on the level of impairment of olfaction. A training program is designed which can bridge basic olfaction skills and develop a course that has sophisticated olfaction tests. In one course, a subject is first asked basic questions about how to identify a smell. A training period of at least one week validates the subject's basic ability to distinguish, for example, pungent and unpleasant odors from neutral odors and pleasant odors. Once complete, a subject progresses to an intermediate level in which other parameters of olfaction are assessed, such as intensity of odor, and at a faster rate. At the advanced level, a subject is challenged with distinguishing mixtures of odors, odors of similar nature, and intensity of odors in parallel. In some modes, an algorithm is used to prioritize testing of particular odors which may be persistently difficult for a subject to identify.

### Example 7: Therapeutic Effect of Olfaction Device

A subject suffering from challenges to mental well-being can be probed to find therapeutic odors which can alleviate symptoms such as negative thought loops. A subject first undergoes a questionnaire with a set of odors, some of which may have pre-existing meaning for the subject, and others which may be totally foreign and unfamiliar. As a subject undergoes a questionnaire, an olfaction device comprises an algorithm which maps emotional states with corresponding odors. An algorithm can then, when appropriate, provide a therapeutically meaningful scent to a subject, especially during difficult or uncomfortable mental states.

### Example 8: Use of Olfactory Therapy for Disrupting Negative, Obsessive, and Trauma Thought Loops

Thought loops are unfavorable mental phenomenon associated with recurring or persistent negative mental states correlated with several mental illnesses. Olfactory therapy is used to disrupt negative, obsessive, and trauma-associated thought loops. A device provided herein is first adjusted to a subject and the olfaction related senses the subject has within a questionnaire. Once calibrated, a regime of olfaction therapy comprising appropriate sets of volatile odorous compounds for a subject is used, assisting in diverting the subject from negative or unfavorable thought loops.

### Example 9: Use of Timing-Based Mechanism During Olfactory Testing and Therapy

Assessing the mental state or well-being of a subject can be quantitatively described by timing responses of a subject during use of a smell assessment tool. Subjects responding rapidly can be categorized as alert or stimulated, and slower responding subjects can be categorized as sluggish or lethargic. From there, an assessment of which subsequent regime of olfactory stimulation can be optimized by an algorithm is performed, further customizing the user experience. This data can be used long term to guide protocols for reducing, for example, negative thought loops within a mental state by providing specific favorable volatile odorous compounds for a given situation a user is within.

### Example 10: Ease of Use and Odor Stability of Self-Administered Smell Test

Smell tests can be costly, time-consuming, and often need expert administration for use outside of intensive research and specialized clinical environments. To address this need, a self-administered smell test (e.g., SCENTinel test as described in Example 2) was designed that rapidly assesses olfactory functions. The smell test, described herein, uses a peel-and-burst technology (e.g., peel and smell technology), with polymeric films. The smell test, described herein (e.g., as shown or described in FIGS. 1A-1C, and/or FIGS. 2-5), can be fully completed online by accessing a survey using a QR code or a web address. Dedicated online REDCap surveys have been implemented to support the proposed remote administration of smell training interventions. To date, more than 5,000 individuals have successfully used the smell test, described herein (e.g., SCENTinel). The smell test, described herein has been mailed all over the US and data has been collected repeatedly from the same participants over 17 weeks (mean±sd: 18±15 times).

### Participants

To assess user experience, a total of 37 adult smell test participants reported on user experience for the odor delivery system (e.g., the smell test, described herein) and the electronic survey (e.g., REDCap surveys) using the User Experience Questionnaire (UEQ). To assess user experience with regard to odor stability, a total of 8 adult smell test participants, with normal olfactory function, reported daily on a 0-100 scale on intensity of the same odor label over a 14-day period.

### Results

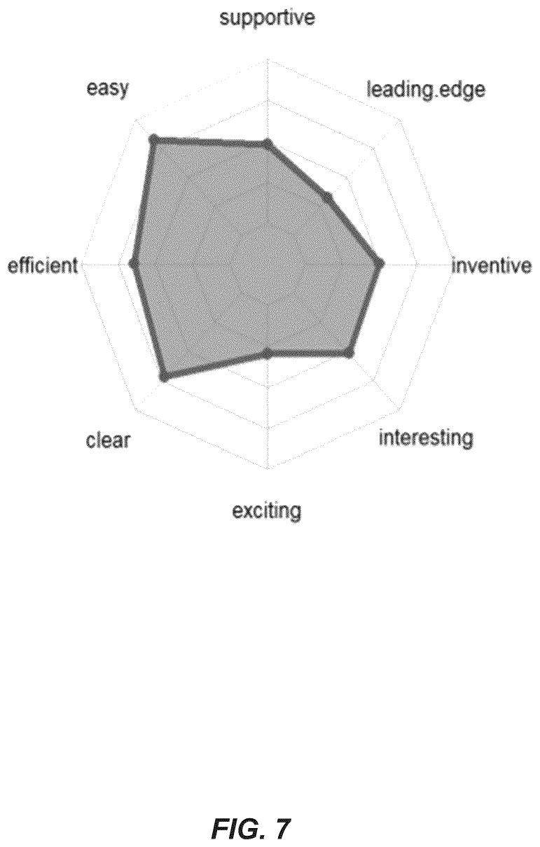

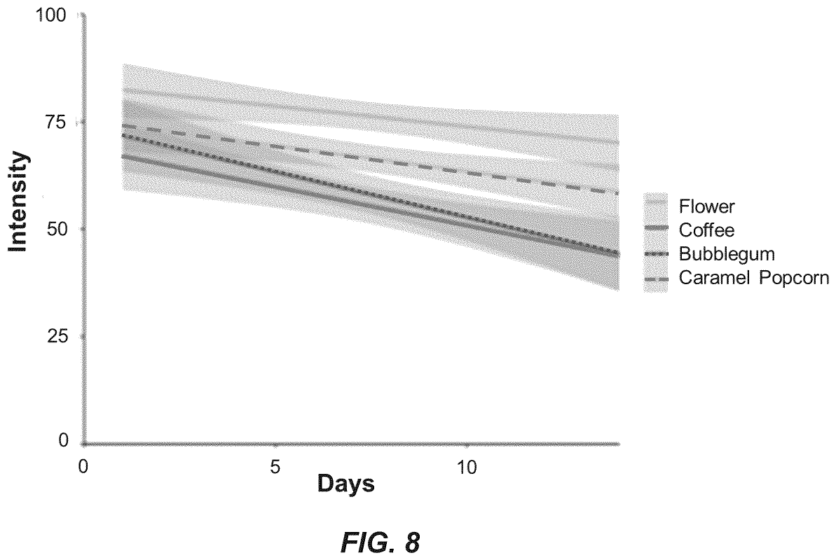

Procedures and analyses were carried out similar to those described in Example 1. Based on standard user experience (UX) criteria, measured via the User Experience Questionnaire (UEQ), the odor delivery system (e.g., the smell test, described herein) and the electronic survey (e.g., REDCap surveys) are perceived as easy, efficient and clear (FIG. 7). The smell test, described herein also shows stability of odor intensity, of the same odor label, as reported daily by users, maintaining odors at stable intensities during mailing and over a 14-day reuse period (FIG. 8). Additionally, anecdotal reports with users up to age 75 indicate ease of use and amusement in taking the smell test, described herein (e.g., SCENTinel).

### Example 11: Use of Olfactory Therapy to Promote Weight Loss

Olfactory strategies can be used as a form of olfactory therapy to help improve weight loss. Odors can take a direct route to the limbic system, which includes the amygdala and hippocampus, key regions involved in emotion and memory processing. Additionally, food odors can be powerfully rewarding cues, including for individuals with obesity, and can act as a stimulus capable of enhancing appetite or promoting satiety for specific foods. Available evidence indicates that short and often unconscious exposure to food odors triggers sensory—specific appetite (SSA), a phenomenon where appetite for a specific food increases and people choose to consume food items that match the specific food (e.g., sweet vs. savory foods). However, a prolonged (e.g., a 20-min exposure or shorter exposures over 5 consecutive days) and conscious exposure to food odors triggers olfactory—specific satiety (OSS), a phenomenon where pleasantness for the matching food decreases, and hence the intake of foods of similar flavor profile (e.g., sweet) diminishes.

By leveraging SSA, the presentation of odors of healthy food items, such as fresh food and vegetables (e.g., orange, strawberry, etc.), with an “olfactory nudge” (ON) (e.g., short exposure over short time frame) may influence people to eat healthy foods. Correspondingly, leveraging OSS, a sustained conscious exposure to indulging food odors (e.g., chocolate) can promote “olfactory habituation” (OH) to that odor, leading to reduced intake of matching food items. This idea is embedded in the incentive-sensitization theory, which suggests amplification of psychological ‘wanting’ (e.g., the motivation to eat energy-dense and low-nutritional value food), with smell cues without matching amplification of ‘liking’ (e.g., the hedonic reaction to eating such foods in obesity). The interaction between these forms of wanting and liking maintains the smell-triggered vicious cycle of unhealthy eating (see, for example, Berridge KC. “Liking” and “wanting” food rewards: brain substrates and roles in eating disorders. Physiol Behav. 2009; 97(5):537-550, which is herein incorporated by reference in its entirety).

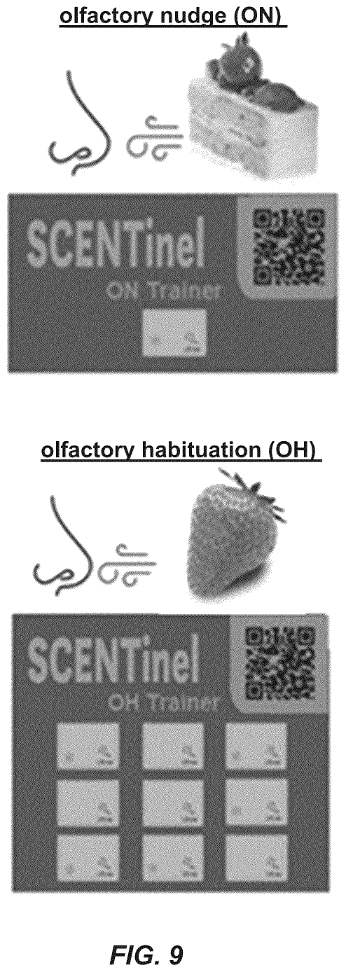

To determine if the two above-proposed olfactory strategies (e.g., olfactory nudge (ON) and olfactory habituation (OH)) can successfully promote weight loss, smell tests, described herein (e.g., SCENTinel) for olfactory nudge (ON) and olfactory habituation (OH) were developed (see FIG. 9 for examples of cards for odor delivery for the ON and OH conditions). Odor stimulation for the ON strategy can be high-energy-dense and low nutritious food (i.e., strawberry cake) and for the OH strategy low-energy dense and high nutritious food (i.e., fresh fruit).

### Participants

To assess whether the two olfactory strategies proposed (e.g., ON and OH) can successfully promote weight loss, a total of 7 adult smell test participants reported on weight loss over a 14-day period. The participants were assessed using olfactory nudge strategy (e.g., smell tests, described herein for odor delivery for the ON condition), olfactory habituation strategy (e.g., smell tests, described herein for odor delivery for the OH condition), or no odor (e.g., control group). The ON strategy had a total of 3 adult smell test participants, the OH strategy had a total of 2 adult smell test participants, and the control group had a total of 2 adult smell test participants.

### Results

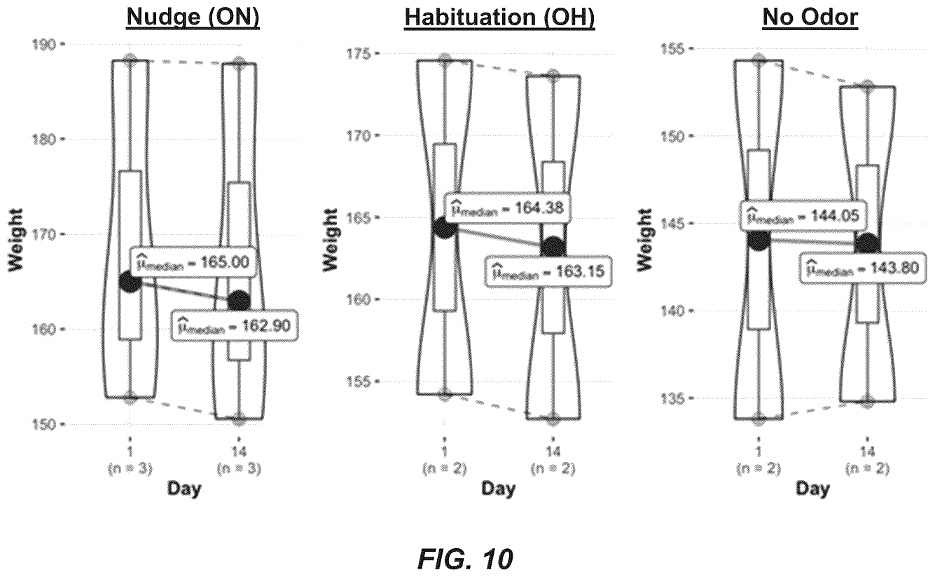

Procedures and analyses were carried out similar to those described in Example 1. Following a dual-process model, use of the smell tests, described herein, demonstrate that the two olfactory strategies proposed (e.g., ON and OH) successfully promote weight loss (FIG. 10). Accordingly, each pilot participant in the ON and OH group lost weight whereas no change is present in the control group (FIG. 10), where n is equal to the number of pilot participants. In some cases, ON and OH can successfully promote weight loss by stimulating automatic engagement in healthy eating and dampening automatic unhealthy eating, respectively. Indeed, individuals in the ON group show a 2-point reduction post-treatment during a Self-Report Behavioral Automaticity Index (SBAI), while those in OH show no difference, and those in the control group increase their automatic responses by 4.5/28 points. In some cases, the olfactory strategies, such as ON, can operate on automatic processes. In contrast, the EVO components (e.g., diet self-monitoring, goal setting, and calorie counting) may primarily rely on reflective processes. The combination of both sets of processes (e.g., olfactory strategies and EVO components) may boost the effectiveness of weight loss programs. Based on these preliminary results, these olfactory strategies (e.g., ON and OH) can support weight loss in obesity.

### Example 12: Enhancing Olfactory Awareness Based on Olfactory Exposure

As described above, olfactory components can promote healthy eating (ON) and discourage unhealthy eating (OH) using regulation via automatic processes by tapping into SSA and OSS, respectively. However, there are substantial individual differences in odor awareness, the ability of spontaneously noticing odors in the environment, that may interfere with the automatic processing of odors. Therefore, participants from the olfactory strategies study (e.g., ON and OH study, Example 11) were also assessed for olfactory awareness based on olfactory exposure.

### Results

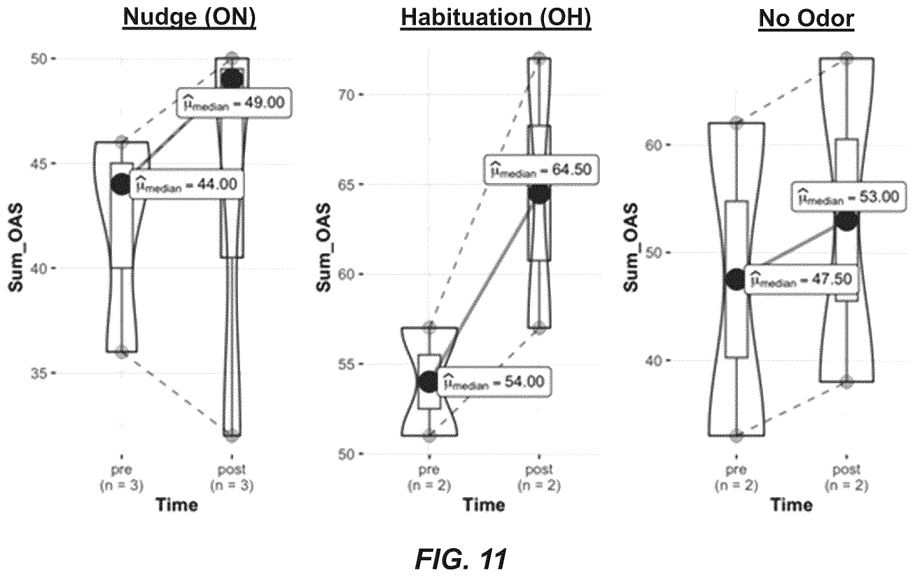

Procedures and analyses were carried out similar to those described in Example 1. Participants are as described in Example 11. Use of the smell tests, described herein, demonstrate that attention to smelling, even if there is no odor, like for those in the control condition, significantly increases odor awareness at the end of the 2-week intervention (V Wilcoxo=26.5, p=0.04, n pairs=7), with greater increases in those in the OH group (10.5±6.4 point increase on a 100-point scale) and no difference in the ON group (1.7±4.9 point increase, FIG. 11). Therefore, in some cases, attention to smelling can increase odor awareness. In some cases, pairing olfactory strategies with evaluative conditioning can boost motivation for adherence to intervention.

### Example 13: Promoting Implementation of Olfactory Therapies

Interventions that are developed with user involvement, as opposed to a top-down expert driven approach, may more likely be feasible, acceptable, and ultimately implementable (see, for example, Mohr et al. Accelerating Digital Mental Health Research From Early Design and Creation to Successful Implementation and Sustainment. J Med Internet Res. 2017; 19(5):e153, which is herein incorporated by reference in its entirety). Therefore, in some cases, engaging users can promote feasibility and acceptability of novel intervention components, such as implementation of olfactory therapies.

### Results

Procedures and analyses were carried out similar to those described in Example 1. Participants are as described in Example 11. Self-reported post-intervention metrics from the olfactory strategies study (e.g., ON and OH study, Example 11) indicate that participants find the ON and OH strategies to be highly acceptable, pleasant and low effort (Table 1). Additionally, participants reported confidence in completing the task (e.g., ON and OH study), and reported a change in thought process about eating, as well as eating behavior (Table 1).

TABLE 1
Post-intervention metrics self-reported on
a 0-5 scale by olfactory exposure group.

| | ON | OH | Control |
|---|---|---|---|
| Liking of task | 4.3 | 3.0 | 3.0 |
| Effort of task | 2.3 | 3.0 | 2.0 |
| Satisfaction with task completion | 4.7 | 4.5 | 3.5 |
| Change in food-related thoughts | 3.7 | 1.5 | 2.0 |
| Change in food-related eating | 3.3 | 1.5 | 2.0 |
| Help with eating healthy | 3.3 | 1.5 | 2.0 |
| Confidence to complete task for 2 weeks | 4.3 | 4.0 | 4.0 |
| Confidence to complete task for 12 weeks | 3.3 | 1.5 | 2.0 |
| Acceptability of task | 4.3 | 3.0 | 3.0 |

### Example 14: Effect of Distinct Odorous Compounds on a Patient's Negative Mental State

As described in Example 8, a device provided herein can be adjusted to a patient according to the results of a questionnaire on olfaction related senses (e.g., patient specific SCENTinel device). Once calibrated, a regime of olfaction therapy comprising appropriate sets of volatile odorous compounds for the patient can be used to help divert negative or unfavorable thought loops. The following case study provides an example of the effect of distinct odorous compounds on a patient's negative mental state.

A Case Study was performed involving a 32-year-old female experiencing anxiety and a negative mental state to determine the effect of distinct odorous compounds on a patient's negative mental state.

### Background and Presenting Complaints

A client of practitioner B. H. (RN, BSN, and MSN), named “J” who is female, 32 years old, a former competitive high school and college athlete, trained social worker, and Reiki student had been experiencing higher levels of anxiety and stress beginning in early 2022. J experienced two significant events during Covid-19: a relationship break-up and a departure from her career as a social worker due to feeling unfulfilled. The events resulted in J's feelings of not being accepted and of unworthiness, accompanied by stress. Through sessions with practitioner B. H., it was revealed that a level of anxiety was present in much of J's life but the life changes during Covid-19 seemed to surface new levels of anxiety. Practitioner B. H. has been conducting healing touch sessions with J periodically since early 2022. Practitioner B. H. is a practicing healing touch provider (HTP). Healing touch (HT) is an energy-based therapeutic approach to healing that emphasizes caring for the whole person.

### History

A medical intake was completed at the initial session. No medical conditions, no hospitalizations, no surgeries and no mental health conditions were reported by J. J takes no prescription or over the counter drugs.

Practitioner B. H. conducted a Body Centered Interview (BCI) which uncovered a prior left wrist fracture (young childhood injury); athletic repetitive-use, over-training and strained muscle injuries; severe constipation (never addressed with a clinician); a history of headaches; and sore feet (from waitressing). Additionally, the BCI revealed that J frequently experienced feeling under pressure during high school related to her athletic performance and significant emotions during college surrounding self-doubt. J reported a feeling of a lump in her throat linked to feelings of being restricted in what she could say. During the BCI, J reported that she could feel practitioner B. H.'s energy.

### Treatment Plan

During the initial session, J reported feeling a lot of anxiety around what choices to make. She felt it was very hard for her to make decisions for herself and plan her own time now that she was not in a relationship where many decisions were made for her.

J wanted to accomplish the following:
a) J wanted to decrease negative thoughts and feel more unconditional love;
b) J wanted to feel calmer—her “brain and energy were feeling frazzled”; and
c) J wanted to stop feeling “ungrounded and overworked”.

J was already practicing self-care including journaling, yoga and meditation. To help alleviate undesirable emotions and physical discomfort that was highlighted during the BCI, J would add the following activities to her self-care routine:
a) a bi-weekly 20-minute journaling session about feeling the “lump in my throat”; and
b) hip stretches using yogic techniques.

During the course of J's Healing Touch sessions, a stressful incident was augmenting J's anxiety and she was offered a specially developed odorous volatile compound (e.g., the smell test, described herein) with instructions to sniff it during feelings of stress and anxiety. Specifically, when J is experiencing negative feelings of stress, she should sniff the odor and try to determine what the smell was. The intention was to distract J from the stress feeling even for a very short period (seconds) in order to facilitate moving on to another mental state.

### Assessment & Progress

J has developed a habit of sniffing the odor (e.g., the smell test, described herein) when stressed and reports that her brain associates it with calm. When she wants to feel calmer, her habit is to reach for the odor provided by practitioner B. H. She reports “her smell” has an earthy, light and citrusy quality. The earthy odor makes her feel grounded.

J finds it hard to describe what the actual smell is and she is not sure if she has trained herself to reach for it to attain feeling calm; the focus on the mystery of the true odor source may be helping her to distract from stressful or negative thoughts. J reports that she likes to use it at the beginning of the day to help her focus. She has been using the smell method to manage stress and negative feelings since September 2023.

Practitioner B. H. conducted a distance healing session with J on Nov. 27, 2023 and noted that J was experiencing some anxiety about a new relationship (feeling a lot of responsibility for him) which was the reason for the session. Generally, she is moving toward a more trust and less anxiety mental state but this situation interrupted that progress.

### Energy Healing Outcome

J is continuing her selfcare plan with “her smell” (e.g., the smell test, described herein) incorporated into a regular self-care plan and reports that the smell is helping to maintain a state of calmness.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-98. (canceled)

99. A method of modulating a user's mental state, the method comprising:
a) providing to the user a panel comprising a patch comprising an odorous volatile compound having a prescribed distinct odor;
b) receiving an input from the user, wherein the input comprises a rating on an intensity of the prescribed distinct odor as perceived by the user; and
c) recording the user's ability to characterize the prescribed distinct odor.

100. The method of claim 99, wherein the panel further comprises a label or image on, or adjacent to, the patch, wherein the image or the label is indicative of the prescribed distinct odor;

101. The method of claim 99, wherein the user has post-traumatic stress disorder, anxiety, depression, disordered eating, or a negative mental state.

102. The method of claim 99, wherein the method influences an eating decision.

103. The method of claim 99, further comprising determining whether the user's mental state has improved after the receiving the input from the user.

104. The method of claim 103, further comprising receiving an additional input from the user, the additional input comprising:
(a) input on the user's physiological data,
(b) input on the user's mood,
(c) input of the user's mental health status, or
(d) a combination of any one thereof.

105. The method of claim 103, wherein the user senses the prescribed distinct odor, wherein sensing the prescribed distinct odor reduces symptoms of a negative mental state in the user.

106. The method of claim 99, further comprising administration or recommendation of a therapeutic agent to the user.

107. The method of claim 99, further comprising assessing a user's time lapse in correctly characterizing the prescribed distinct odor.

108. A smell presentation system, comprising:
a) a plurality of panels, each panel comprising:
i) an odorous volatile compound having a prescribed distinct odor;
b) an electronic system comprising:
i) an output device that prompts a user to sense, by olfaction, the odorous volatile compounds, and
ii) an input device that registers input from the user when the user attempts to identify the odorous volatile compound, wherein the input comprises a rating of the prescribed distinct odor.

109. The smell presentation system of claim 108, wherein the panel further comprises an image or label located adjacent to the odorous volatile compound or on a panel cover, wherein the image or the label is indicative of the prescribed distinct odor of the odorous volatile compound.

110. The smell presentation system of claim 108, wherein the electronic system further comprises a storage device that stores the input.

111. The smell presentation system of claim 108, wherein the input device obtains the input from the user on whether the user senses, by olfaction, each of the odorous volatile compound.

112. The smell presentation system of claim 111, wherein the electronic system compares the input to a reference.

113. The smell presentation system of claim 108, further comprises a processor that determines whether the user's mental state improves over time.

114. The smell presentation system of claim 108, wherein the electronic system further provides a psychological questionnaire to the user.

115. The smell presentation system of claim 108, wherein the electronic system further receives additional input from the user, the additional input comprises:
(a) input on the user's physical health,
(b) input on the user's mood,
(c) input of the user's mental health status, or
(d) a combination thereof.

116. The smell presentation system of claim 108, wherein the user has post-traumatic stress disorder, anxiety, depression, or disordered eating.

117. The smell presentation system of claim 108, wherein a prompt by the output device is gamified.

118. The smell presentation system of claim 108, wherein the smell presentation system influences an eating decision.
